(19) United States
(12) Patent Application Publication
(10) Pub. No.: US 2022/0175276 A1
(43) Pub. Date: Jun. 9, 2022

(54) HEALTH AND FITNESS RECOMMENDATION SYSTEMS

(71) Applicant: Key Point Technologies Inc., Hermosa, CA (US)

(72) Inventors: Paige Sullivan, Hermosa Beach, CA (US); Kinglsey McGowan, Houston, TX (US)

(21) Appl. No.: 17/111,118

(22) Filed: Dec. 3, 2020

(51) Int. Cl. A61B 5/11; G16H 20/30; A61B 5/00; G16H 20/60; G16H 20/70

(52) U.S. Cl. CPC A61B 5/1118; G16H 20/30; A61B 5/1112; G16H 20/70; G16H 20/60; A61B 5/0022; A61B 5/6801

(57) ABSTRACT

A recommendation system may store user data, fitness class data, and expert rules data. The user data may correspond to health or fitness information about a user. The fitness class data may correspond to information about a fitness class. The expert rules data may correspond to an action for accomplishing a health objective. The recommendation system may be configured to determine health objective data based on the user data. The health objective data may be determined directly or inferentially. The health objective data may be matched with the expert rules data. The expert rules data may be matched with the fitness class data. The fitness class data may correspond to an aspect of the fitness class that aligns with the health objective of the user. Recommendation data may be generated that corresponds to a recommendation of the fitness class for the user.

### BACKGROUND

The health and fitness industry has exploded with options for individuals to manage their personal health and fitness. Many of those options incorporate technology in various ways. However, the increase of technology has not diminished demand for social health and fitness settings; rather, technology may be at least partly responsible for an increase in demand for social options such as health and fitness classes. Businesses and software have started popping up to address this demand. Indeed, the health and fitness market has become so inundated with options that consumers frequently experience choice paralysis. The plethora of options and sometimes conflicting advice can lead consumers to engage in incorrect or ineffective activities. In turn, consumers may fall short of their ideal health and fitness.

### BRIEF DESCRIPTION OF THE DRAWINGS

The present description will be understood more fully when viewed in conjunction with the accompanying drawings of various examples of health and fitness recommendation systems. The description is not meant to limit the health and fitness recommendation systems to the specific examples. Rather, the specific examples depicted and described are provided for explanation and understanding of health and fitness recommendation systems. Throughout the description the drawings may be referred to as drawings, figures, and/or FIGS.

FIG. 1 illustrates a health and fitness device network, according to an embodiment.

FIG. 2 illustrates a recommendation system, according to an embodiment.

FIG. 3 illustrates a cloud-based recommendation system, according to an embodiment.

FIG. 4 illustrates a map displaying geolocation for a user and a fitness class, according to an embodiment.

FIG. 5A illustrates the user device displaying a user interface where a user can input information about the user, according to an embodiment.

FIG. 5B illustrates the user device displaying a user interface where the user can input fitness information about the user, according to an embodiment.

FIG. 5C illustrates the user device displaying fitness information about the user on the user interface, according to an embodiment.

FIG. 5D illustrates the user device displaying information about a goal of the user on a user interface, according to an embodiment.

FIG. 6A illustrates a web site where the recommendation system may source fitness class data, according to an embodiment.

FIG. 6B illustrates a mobile device application where the recommendation system may source fitness class data, according to an embodiment.

FIG. 7 illustrates a method of generating a fitness class recommendation for a user, according to an embodiment.

FIG. 8 illustrates another method of generating the fitness class recommendation, according to an embodiment.

FIG. 9 illustrates a method of using instructor and/or class rating data to recommend the fitness class, according to an embodiment.

FIG. 10 illustrates a method of using a correlative filter to recommend the fitness class, according to an embodiment.

FIG. 11 illustrates a method of recommending the fitness class based on data obtained from a third-party fitness class data resource, according to an embodiment.

FIG. 12 illustrates a method of recommending the fitness class by matching the fitness class data and the user data to corresponding rule data, according to an embodiment.

FIG. 13 illustrates a method of recommending a recovery period, according to an embodiment.

### DETAILED DESCRIPTION

Health and fitness recommendation systems as disclosed herein will become better understood through a review of the following detailed description in conjunction with the figures. The detailed description and figures provide merely examples of the various embodiments of health and fitness recommendation systems. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

A conventional health or fitness software application may compile information about an individual. The information may be provided by the individual or collected using a device such as an activity tracker, a health monitor, and so forth. Another conventional health or fitness software application may compile information about fitness classes. The fitness classes may be in-person classes or online classes. The information may be provided for reference by the individual. The information may include links to download and/or stream online fitness classes. Yet another conventional health or fitness software application may provide nutrition information, such as for weight-loss. As one example, an individual may accrue points that correspond to the nutritional value of food the individual has consumed. This may be helpful up to a certain point, but the shortcomings point clearly to the need for a better solution.

Applications that compile health and fitness information about an individual are informative but not insightful. The individual may know what her current health or fitness status is but may not know what to do with that information. Applications that compile fitness class information may similarly provide information without insight. The individual may become overwhelmed by the number of fitness class choices. The individual may have no way to determine which class to take and may end up taking the wrong class or no class at all. Applications that provide nutrition information may be disconnected from an individual's fitness regime and/or fitness goals. This may hinder the individual from accomplishing her fitness goals.

Implementations of health and fitness recommendation systems as described below may address some or all of the problems described above. A health and fitness recommendation system may include a database server and a processing server. The database server may store a fitness knowledge database. The fitness knowledge database may include fitness class data and fitness outcome data. The fitness class data may correspond to information about a fitness class. The fitness class data may include class location data that corresponds to a location of the fitness class. The fitness outcome data may correspond to a fitness outcome associated with the fitness class.

The database server may store a user knowledge database. The user knowledge database may include health and fitness data that corresponds to health and fitness information about a user. The health and fitness data may include user location data that corresponds to a location of the user. The health and fitness data may include location range data that corresponds to a maximum distance preferred by the user for a distance between the location of the user and the location of the fitness class. The health and fitness data may include fitness goal data that corresponds to a fitness goal of the user.

The processing server may be communicatively coupled to the database server. The processing server may include a fitness inference engine. The fitness inference engine may be configured to determine whether a difference between the class location data and the user location data is reflected in the location range data. The fitness inference engine may be configured to output touchpoint data. An individual touchpoint may include a threshold level of commonality or a threshold degree of alignment between the fitness class data and the health and fitness data. The individual touchpoint may include a threshold level of commonality or a threshold degree of alignment between the fitness outcome data and the health and fitness data. The individual touchpoint may include a threshold level of commonality or a threshold degree of alignment between or the fitness class data, the fitness outcome data, and the health and fitness data.

The fitness inference engine may be configured to output recommendation data. The recommendation data may correspond to a recommendation of the fitness class for the user. The fitness class may be recommended for the user in response to the difference between the class location data and the user location data being reflected in the location range data. The fitness class may be recommended for the user in response to the touchpoint data including a threshold quantity of individual touchpoints satisfying the threshold level of commonality or the threshold degree of alignment.

As another example, a health and fitness recommendation system may include a memory component. The memory component may be configured to store user data. The user data may include user health data that corresponds to health information about a user. The user data may include user fitness data that corresponds to fitness information about the user. The memory component may be configured to store fitness class data that corresponds to information about a fitness class. The memory component may be configured to store expert rules data that corresponds to an action for accomplishing a health objective.

The health and fitness recommendation system may include a processing component in communication with the memory component. The processing component may be configured to output health objective data based on the user data. The health objective data may be determined, based on the user data, directly or inferentially. The processing component may be configured to match an element of the health objective data with a corresponding element of the expert rules data. The element of the health objective data and the element of the expert rules data may be matched directly or inferentially. The processing component may be configured to match the element of the expert rules data with an element of the fitness class data. The fitness class data may correspond to an aspect of the fitness class that aligns with the health objective of the user based on the expert rules data. The element of the expert rules data and the element of the fitness class data may be matched directly or inferentially. The processing component may be configured to output recommendation data that corresponds to a recommendation of the fitness class for the user.

Health and fitness recommendation systems as described herein may provide insight to individuals overwhelmed by the amount of information and number of options available for health and fitness. For example, whereas conventional technologies may simply provide options for various fitness classes, the recommendation systems as described herein may provide specific guidance tailored to an individual based on that individual's current health and/or fitness and the individual's health and/or fitness goals. The guidance may be based on rules established by experts in the fields of health and fitness. By pointing the individual to specifically-selected classes based on expert guidance, the barrage of options is defrayed into a clear path by which the individual can accomplish specific goals.

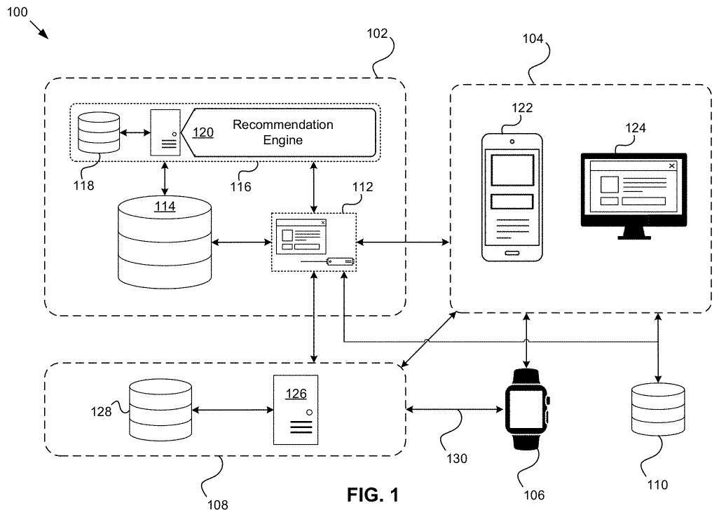

FIG. 1 illustrates a health and fitness device network 100, according to an embodiment. The health and fitness device network 100 may enable an individual to obtain insightful recommendations that are personalized for the individual based on expert advice. The recommendations may be obtained by the individual without incurring the sometimes significant costs associated with hiring a personal fitness trainer or coach. The recommendations may be based on the individual's personal health and fitness status, the individual's goals, where the individual is located, and what health and fitness resources the individual has access to. This insight at low cost may increase the availability of highly-tailored health and fitness information to populations that previously may not have had access to such resources.

The health and fitness device network 100 may include a recommendation system 102, a user device 104, a wearable device 106 (i.e. a wearable device), a wearables server 108, and a fitness class server 110. The recommendation system 102 may include an application server 112, a data server 114, and a recommendation server 116. The recommendation server 116 may include an expert rules database 118 and a recommendation engine 120. The user device 104 may include, as examples, a smartphone 122 or a personal computer 124. The wearables server 108 may include a wearables application server 126 and a measurement database 128. Devices in the health and fitness device network 100 may be communicatively coupled to each other by one or more communication links 130.

The health and fitness device network 100 may be and/or include a body area network (BAN), a personal area network (PAN), a near-me area network (NAN), a local area network (LAN), a metropolitan area network (MAN), a wide area network (WAN), an internet area network (IAN), and/or a public Internet network. The health and fitness device network 100 may include two or more network types. For example, the health and fitness device network 100 may include the BAN, the LAN, and the Internet. In another example, the health and fitness device network 100 may include the NAN, the MAN, and the Internet.

The health and fitness device network 100 may include a point-to-point topology, a daisy chain topology, a bus topology, a star topology, a ring topology, and/or a mesh topology. The health and fitness device network 100 may include a hybrid topology including two or more types of network topologies. For example, the health and fitness device network 100 may include a mesh topology and a star topology. In another example, the health and fitness device network 100 may include a point-to-point topology and a star topology.

The term “home” may refer to a user and/or individual's residence. The term “metropolitan” may refer to a geographic region having homes, buildings, offices, and/or campuses. The term “remote” may refer to a device being accessible by another device via a network communication link such as the communication link 130. The term “remote” may also refer to non-adjacent locations. For example, two non-adjacent rooms within a building may be considered remote from each other, two non-adjacent buildings may be considered remote from each other, two non-adjacent metropolitan areas may be considered remote from each other, and so forth. The term “local” may refer to a device being connected via a closed and/or private network connection to another device. The term “local” may also indicate physical location within a same room, within a same building, on a same campus, and/or in a same metropolitan area, and so forth.

The health and fitness device network 100 may be situated in a single location, such as in a room, in a user's home, within an office building, and/or within a campus. For example, all networked elements of the health and fitness device network 100 may be physically located in the same room as each other, in the user's home, within an office building, and/or on the same campus. In various other embodiments, the health and fitness device network 100 may be situated across two or more locations, such as across two rooms, between a user's home and an office building, across buildings on different campuses from each other, across different metropolitan areas, and so forth.

For example, some of the networked elements of the health and fitness device network 100 may be situated in the user's home, and some of the networked elements may be situated in an office building housing a data center in a different city and/or country from the user's home. In another example, some of the networked elements may be situated in a data center, some of the networked elements may be situated in another data center, and some of the networked elements may be mobile, accompanying the user as the user moves and travels from one location to another. Some of the networked elements may be situated in a cloud-based networking system.

The communication links 130 may be direct or indirect. A direct link may include a link between two devices where information is communicated from one device to the other without passing through an intermediary. For example, the direct link may include a Bluetooth™ connection, a Zigbee® connection, a Wifi Direct™ connection, a near-field communications (NFC) connection, an infrared connection, a wired universal serial bus (USB) connection, an ethernet cable connection, a fiber-optic connection, a firewire connection, a microwire connection, and so forth. In another example, the direct link may include a cable on a bus network. “Direct,” when used regarding the communication links 130, may refer to any of the aforementioned direct communication links.

An indirect link may include a link between two or more devices where data may pass through an intermediary, such as a router, before being received by an intended recipient of the data. For example, the indirect link may include a wireless fidelity (WiFi) connection where data is passed through a WiFi router, a cellular network connection where data is passed through a cellular network router, a wired network connection where devices are interconnected through hubs and/or routers, and so forth. The cellular network connection may be implemented according to one or more cellular network standards, including the global system for mobile communications (GSM) standard, a code division multiple access (CDMA) standard such as the universal mobile telecommunications standard, an orthogonal frequency division multiple access (OFDMA) standard such as the long term evolution (LTE) standard, and so forth. “Indirect,” when used regarding the communication links 130, may refer to any of the aforementioned indirect communication links.

Various of the devices in the health and fitness device network 100 may include data storage and/or processing capabilities. Such capabilities may be rendered by various electronics for processing and/or storing electronic signals. One or more of the devices in the health and fitness device network 100 may include a processing device. For example, the wearable device 106, the application server 112, the recommendation server 116, the smartphone 122, the personal computer 124, and/or the wearables application server 126 may include the processing device. One or more of the devices in the health and fitness device network 100 may include a memory device. For example, the wearable device 106, the fitness class server 110, the data server 114, the expert rules database 118, the smartphone 122, the personal computer 124, and/or the measurement database 128 may include the memory device.

The processing device may have non-transitory and/or transitory memory. The memory device may have non-transitory and/or transitory memory. The processing device may have transitory memory and the memory device may have persistent memory. The processing device may generate an output based on an input. For example, the processing device may receive an electronic and/or digital signal. The processing device may send the signal to the memory device, and the memory device may store associated with the signal. The processing device may read the signal and perform one or more tasks with the signal, such as performing various functions with data in response to input received by the processing device. The processing device may read from the memory device information needed to perform the functions. For example, the processing device may make a recommendation based on a received input and a rule stored on the memory device.

The processing device may be and/or include a processor, a microprocessor, a computer processing unit (CPU), a graphics processing unit (GPU), a neural processing unit, a physics processing unit, a digital signal processor, an image signal processor, a synergistic processing element, a field-programmable gate array (FPGA), a sound chip, a multi-core processor, and so forth. As used herein, “processor,” “processing component,” “processing device,” and/or “processing unit” may be used generically to refer to any or all of the aforementioned specific devices, elements, and/or features of the processing device.

The memory device may be and/or include a computer processing unit register, a cache memory, a magnetic disk, an optical disk, a solid-state drive, and so forth. The memory device may be configured with random access memory (RAM), read-only memory (ROM), static RAM, dynamic RAM, masked ROM, programmable ROM, erasable and programmable ROM, electrically erasable and programmable ROM, and so forth. As used herein, “memory,” “memory component,” “memory device,” and/or “memory unit” may be used generically to refer to any or all of the aforementioned specific devices, elements, and/or features of the memory device.

Various of the devices in the health and fitness device network 100 may include data communication capabilities. Such capabilities may be rendered by various electronics for transmitting and/or receiving electronic and/or electromagnetic signals. One or more of the devices in the health and fitness device network 100 may include a communication device. For example, the wearable device 106, the fitness class server 110, the application server 112, the data server 114, the recommendation server 116, the expert rules database 118, the smartphone 122, the personal computer 124, and/or the wearables application server 126 may include the communication device.

The communication device may include, for example, a networking chip, one or more antennas, and/or one or more communication ports. The communication device may generate radio frequency (RF) signals and transmit the RF signals via one or more of the antennas. The communication device may receive and/or translate the RF signals. The communication device may transceive the RF signals. The RF signals may be broadcast and/or received by the antennas.

The communication device may generate electronic signals and transmit the RF signals via one or more of the communication ports. The communication device may receive the RF signals from one or more of the communication ports. The electronic signals may be transmitted to and/or from a communication hardline by the communication ports. The communication device may generate optical signals and transmit the optical signals to one or more of the communication ports (e.g. by a fiber-optic communication system).

The communication device may include hardware and/or software for generating and communicating signals over a direct and/or indirect network communication link. For example, the communication component may include a USB port and a USB wire, and/or an RF antenna with Bluetooth™ programming installed on a processor, such as the processing component, coupled to the antenna. In another example, the communication component may include an RF antenna and programming installed on a processor, such as the processing device, for communicating over a Wifi and/or cellular network. As used herein, “communication device” “communication component,” and/or “communication unit” may be used generically herein to refer to any or all of the aforementioned elements and/or features of the communication component.

Various of the devices in the health and fitness device network 100 may be referred to as a “server.” Such devices may include a server device. The server device may include a physical server and/or a virtual server. For example, the server device may include one or more bare-metal servers. The bare-metal servers may be single-tenant servers or multiple tenant servers. In another example, the server device may include a bare metal server partitioned into two or more virtual servers. The virtual servers may include separate operating systems and/or applications from each other. In yet another example, the server device may include a virtual server distributed on a cluster of networked physical servers. The virtual servers may include an operating system and/or one or more applications installed on the virtual server and distributed across the cluster of networked physical servers. In yet another example, the server device may include more than one virtual server distributed across a cluster of networked physical servers.

The term server may refer to the functionality of a device. For example, an application server may be programming instantiated in an operating system that receives, retrieves, stores, outputs, and/or processes data. A recommendation server may be programming instantiated in an operating system the correlates data, applies rules to data to make inferences about the data, and so forth. Servers referred to separately herein, such as an application server and a recommendation server, may be instantiated in the same operating system and/or on the same server device. Separate servers may be instantiated in the same application or in different applications.

Various aspects of the systems and networks described herein may be referred to as “data.” Data may be used to refer generically to modes of storing and/or conveying information. Accordingly, data may refer to textual entries in a table of a database. Data may refer to alphanumeric characters stored in a database. Data may refer to machine-readable code. Data may refer to images. Data may refer to audio. Data may refer to, more broadly, a sequence of one or more symbols. The symbols may be binary. Data may refer to a machine state that is computer-readable. Data may refer to human-readable text.

The wearable device 106 may be configured to take a measurement from a user. For example, the user may wear the wearable device 106 on a wrist of the user. The wearable device 106 may include a sensor. The sensor may, for example, include an optical device, an impedance device, and so forth. A processor in the wearable device 106 may be configured to take the measurement by the sensor. The measurement may be a physiological measurement (e.g. a measurement of a physiological characteristic of the user). The processor of the wearable device 16 may be configured to generate measurement data based on the measurement taken from the user. The wearable device 106 may be configured to communicate with the user device 104. The wearable device 106 may be configured to communicate directly with one or more elements of the recommendation system 102. The wearable device 106 may be configured to communicate with the wearables server 108.

The measurement by the wearable device 106 may be and/or include a biometric reading and/or measurement. The biometric reading/measurement may determine biometric information about the user. The measurement data may be and/or include biometry data. The biometry data may be based on the biometric reading/measurement. The measurement may be and/or include an activity reading and/or measurement. The activity reading/measurement may determine an activity engaged in by the user. The measurement data may be and/or include activity data. The activity data may be based on the activity reading/measurement. A recommendation and/or recommendation data as described herein may be based at least in part on the biometric reading or the activity reading.

The wearables server 108 may be communicatively coupled to the wearable device 106 and/or the user device 104. The wearables server may be configured to receive the measurement data from the wearable device 106. The wearables server 108 may be configured to store the measurement data. The wearables server 108 may be configured to preprocess the measurement data (i.e. to clean the measurement data, organize the measurement data, mine the measurement data, and so forth). The wearables server 108 may be configured to communicate the measurement data to a receiving device such as the user device 104, devices in the recommendation system 102, and so forth.

The user device 104 may be communicatively coupled to the recommendation system 102, wearable device 106, the wearables server 108, and/or the fitness class server 110. The user device may be configured to receive the measurement data from the wearable device 106 or the wearables server 108. The user device 104 may be configured to receive an input from a user. For example, the user device 104 may include a user interface. The user interface may display a form that prompts the user to input information into the form.

The user device 104 may be configured to generate user data based on the measurement data or the input from the user. The user data may be and/or include input data that corresponds to the input from the user. The user data may be and/or include user health data. The input data may be and/or include the user health data. The user health data may correspond to health information about the user such as biometric information and/or vitals information. The user health data may correspond to a health goal for the user. The user data may be and/or include user fitness data.

The input data may be and/or include the user fitness data. The user fitness data may correspond to fitness information about the user such as a location of the user (e.g. where the user is located when the user exercises or generally where a user is located when the user would like to exercise), a fitness routine of the ser, a fitness goal of the user, an exercise preference of the user, a self-identified skill level of the user in a fitness class, and so forth. The user fitness data may include information about fitness equipment available to the user, such as fitness equipment in the user's home and/or at a gym the user attends.

The user health data may include biometry data. The biometry data may correspond to the biometric information about the user. The biometric information may include a heart rate variability (HRV) of the user, a resting heart rate of the user, a VO₂ max of the user, and so forth. The HRV of the user may be represented in the biometric data by HRV data. The resting heart rate of the user may be represented in the biometric data by resting heart rate data. The VO₂ max of the user may be represented in the biometric data by VO₂ max data.

The user health data may include vitals data. The vitals data may correspond to demographic vitals of the user. The demographic vitals may include the user's weight, the user's height, the user's age, the user's race and/or ethnicity, the user's gender and/or gender identity, and so forth. The user's weight may be represented in the vitals data by weight data, the user's height may be represented in the vitals data by height data, and so forth.

The user health data may include health goal data. The health goal data may correspond to the health goal for the user. The health goal may be, for example, losing weight, managing a chronic health condition, improving the user's HRV, decreasing the user's resting heart rate, increasing the user's VO₂ max, improving the user's blood pressure, reducing the user's stress level, and so forth.

The user fitness data may include user location data that corresponds to the location of the user. The user fitness data may include location range data that corresponds to a preference of the user for a maximum distance from the location of the user to a location of a fitness class (i.e. an indication of how far the user will travel for a fitness class). The user fitness data may include fitness routine data that corresponds to the fitness routine of the user. The user fitness data may include fitness goal data that corresponds to the fitness goal for the user. The user fitness data may include preference data that corresponds to the preference of the user. The user fitness data may include skill data that corresponds to the self-identified skill level of the user in a particular fitness class. The user fitness data may include activity data. The activity data corresponds to the activity measurement taken by the wearable device 106.

The user location data may include geographic data for the location of the user. The geographic data may include a physical address for the user, e.g. a home or office address for the user. The geographic data may include a latitude and/or longitude where the user is located or was recently located. The geographic data may be an internet protocol address. The user location data may include region data for a geographic region associated with the user. The region data may include a name of the geographic region, such as a city, a county, a borough, and so forth. The region data may include defining terms for a boundary of the region, such as ranges of latitude and longitude or radii from a fixed location. The user location data may include travel data that corresponds to a travel routine of the user. For example, the travel data may include mapping of a route the user takes from home to work. The travel data may correspond to a route the user takes regularly, such as in running errands.

The user location data may include an indication the user has a preference for digital and/or online health and fitness content. The user's physical location and/or IP address may be hidden. The user location data may accordingly be null or empty. The application server 112 and/or the recommendation server 116 may be programmed to recognize the user prefers online and/or digital health and fitness content when the user location data is null or empty.

The fitness routine data may include user exercise data. The user exercise data may correspond to an actual exercise engaged in by the user. For example, the user may engage in running on a regular or semi-regular basis. The user may engage in yoga on a regular basis. The user exercise data may include exercise setting data. The exercise setting data may correspond to a setting in which the user typically exercises. For example, the setting in which the user typically exercises may be at a gym, at home, outdoors, and so forth.

The fitness routine data may include routine day data and/or time of day data. The routine day data may indicate a day and/or days on which the user typically exercises. For example, the routine day data may indicate the user exercises three times per week on Mondays, Wednesdays, and Saturdays. The time of day data may indicate a time of day the user typically exercises. For example, the time of day data may indicate the user typically exercises in the mornings.

The fitness goal data may correspond to a fitness objective for the user. The fitness objective may be specific or general. For example, the user may have a specific fitness objective of increasing his sprinting speed in the forty-yard dash by a specific amount. As another example, the user may have a general fitness objective of becoming more active. The fitness objective may be action-based or outcome-based. For example, the user may have an action-based fitness objective of exercising for thirty minutes five times per week. As another example, the user may have an outcome-based fitness objective of increasing her maximum deadlift weight by twenty pounds.

The preference data may correspond to a preference the user has related to exercise and/or fitness. The preference data may include exercise type data that corresponds to a type of exercise preferred by the user (e.g. the user may prefer weight-lifting over swimming). The preference data may include class type data that corresponds to a type of fitness class preferred by the user (e.g. the user may prefer yoga classes over high-intensity interval training (HIIT) classes). The preference data may include gender preference data. The gender preference data may correspond to a gender preferred by the user for an instructor of a fitness class. The gender preference data may correspond to a predominant gender for attendees of the fitness class.

The preference data may include rating preference data. The rating preference data may correspond to an attendee rating preferred by the user for the instructor (e.g. the user's preference is that attendees of classes taught by a particular instructor give that instructor at least four out of five stars). The rating preference data may correspond to an attendee rating preferred by the user for the fitness class independent of the instructor (e.g. the user's preference is that attendees of a water aerobics class give the class at least four out of five stars). The preference data may include qualification preference data. The qualification preference data may correspond to a qualification preferred by the user for the instructor (e.g. the user's preference is that the instructor is or was a professional athlete). The qualification preference data may be generated by the user device 104 in response to input by the user into the user device 104.

The skill data may correspond to a specific skill characterization or a general skill characterization. For example, the skill data may correspond to a specific skill level in a fitness class (e.g. the user has a black belt in jiu-jitsu). As another example, the skill data may correspond to a general skill and/or fitness level of the user (e.g. the user considers himself to have low aerobic stamina). The skill characterization may be self-identified by the user, such as by input by the user into the user device 104. The skill characterization may be based on an assessment of the user's fitness ability.

The user data may be a combination of the input data and the measurement data. The input data may be and/or include a first element of the user health data. The first element of the user health data may, for example, correspond to a type of information that is provided by the user. For example, the user may input into the form displayed on the user device 104 information about health conditions of the user, such as an indication the user has been diagnosed with asthma, high blood pressure, and so forth.

The input data may be and/or include a first element of the user fitness data. The first element of the user fitness data may, for example, correspond to a type of information that is provided by the user. For example, the user may input into the form displayed on the user device 104 information about the user's preferences for an exercise, a fitness class, an instructor of the fitness class, and so forth.

The user data may be and/or include the measurement data from the wearable device 106 and/or the wearables server 108. The measurement data may be and/or include an element of the user health data. For example, the measurement data may correspond to the HRV of the user, the resting heart rate of the user, the VO₂ max of the user, and so forth. The measurement data may be and/or include an element of the user fitness data. For example, the measurement data may be the routine data, where the measurement data indicates that the user's heart rate is elevated for a period of time with predictable regularity (e.g. the user exercises in the mornings on weekdays).

The user device 104 may include a location device such as a global positioning system device. The wearable device 106 may include the location device. The location device may generate location data that may be included with the user data and/or the measurement data. The location device may be communicatively coupled to the user device 104, the wearable device 106, the wearables server 108, and/or a device in the recommendation system 102. The location device may be configured to output user location data that corresponds to the location of the user.

The user device 104 may be configured to communicate the user data to a receiving device such as a device in the recommendation system 102, the wearable device 106, the wearables server 108, and so forth. The user device 104 may be configured to receive recommendation data from the recommendation system 102. The recommendation data may correspond to a health and/or fitness recommendation for the user. The user device 104 may be configured to display the recommendation based on the recommendation data. For example, the user device 104 may display the recommendation using natural language (i.e. “take fitness class X once per week to accomplish your goal”) on the user interface.

As used herein, fitness class may refer to a fitness activity. The fitness activity may be engaged in with other participants. The fitness activity may be engaged in alone by the user. The fitness activity may be engaged in under supervision and/or instruction by an instructor. The fitness activity may be structured or unstructured.

The fitness class server 110 may be and/or include a website server. The fitness class server 110 may be and/or include internet web pages. The website server and/or the web pages may contain information about a fitness class. For example, the fitness class server 110 may be a web page by a fitness studio that includes types of classes offered at the fitness studio, days and times the classes are offered, information about instructors at the fitness studio, and so forth. As another example, the fitness class server 110 may be a website server that serves web pages with information about a variety of fitness classes offered at a variety of locations.

The fitness class server 110 may be and/or include an application installed on the user device 104. The application may be specific to a particular provider of fitness classes (e.g. an application for a gym). The application may compile fitness class information from a variety of providers.

The fitness class server 110 may be a third-party data resource (i.e. may be owned and/or operated by an entity separate from the user and/or an entity associated with the recommendation system 102). The fitness class server 110 may be owned and/or operated by the user and/or the entity associated with the recommendation system 102. The fitness class server 110 may be a public data resource. For example, the resource may include information accessible to anyone online without requiring login or other authentication information. The fitness class server 110 may be a private data resource. For example, the resource may include information accessible by logging in or otherwise authenticating as an authorized viewer of the information.

The fitness class server 110 may be communicatively coupled to the user device 104. The fitness class server may be configured to store fitness class data. The fitness class data may correspond to the information about the fitness class. The fitness class data may correspond to outcomes associated with the fitness class. The fitness class data may correspond to preference factors associated with the fitness class. The fitness class server 110 may be configured to communicate the fitness class data to the user device 104.

The fitness class data may be and/or include class location data. The class location data may correspond to a location for the fitness class (e.g. a geographic location or an indication the fitness class is online). The fitness class data may be and/or include schedule data. The schedule data may correspond to a schedule for the fitness class. The fitness class data may be and/or include instructor data. The instructor data may correspond to information about an instructor of the fitness class. The fitness class data may be and/or include class exercise data. The class exercise data may correspond to a type of exercise performed in the fitness class. The fitness class data may be and/or include difficulty data. The difficulty data may correspond to a difficulty level of the fitness class.

The class location data may include geographic data for a geographic location of the fitness class. The geographic data may include an address. The geographic data may include latitude and longitude for the location of the class. The class location data may include digital address data. The digital address data may indicate the fitness class is a virtual class on a digital platform. The digital address data may include a uniform resource locator (URL) that specifies a location of files associated with the virtual class in a computer network. The digital address data may include information about live-streaming of the fitness class. The class location data may include the geographic data and the digital address data, which may indicate the fitness class is offered in-person and online.

The schedule data may include class day data that corresponds to a day on which the fitness class is held. The schedule data may include start time data that corresponds to a start time of the fitness class. The schedule data may include duration data that corresponds to a duration of the fitness class. The schedule data may include null data and/or an empty data set that indicates the fitness class can be taken at any time (e.g. the fitness class is streamable from a digital platform). The schedule data may include null data and/or an empty data set that indicates a schedule for the fitness class does not exist. For example, an in-person fitness class may be canceled indefinitely because of a global health pandemic. The null data may indicate there are no planned classes for the fitness class.

The instructor data may include gender data that indicates a gender of the instructor. The instructor data may include rating data that corresponds to a rating for the instructor by attendees of the fitness class. For example, the rating data may include a numerical score for the instructor, such as five out of five stars. The instructor data may include qualification data that corresponds to a qualification of the instructor to instruct the fitness class. For example, the fitness class may be a yoga class and the qualification may be that the instructor has a registered yoga teacher certification from a registered yoga school.

The difficulty data may provide a qualitative indication of the difficulty of the class or a quantitative indication of the difficulty of the class. The difficulty data may include qualitative data that corresponds to a qualitative assessment of the difficulty of the class. For example, the qualitative assessment may be that the fitness class is for beginners. The difficulty data may include quantitative data that corresponds to a quantitative assessment of the difficulty of the class. The quantitative data may include HRV range data that corresponds to a range of suitable HRVs for the fitness class. The quantitative data may include resting heart rate range data that corresponds to a range of suitable resting heart rate values for the fitness class (e.g. where resting heart rate is an indicator of overall fitness of an individual). The quantitative data may include VO₂ max range data that corresponds to a range of suitable VO₂ max measurements for the fitness class.

The recommendation system 102 may be configured to output the health and/or fitness recommendation for the user based at least in part on the user data and/or the fitness class data. The recommendation system 102 may be configured to receive data, store data, process data, and output data. The recommendation system may be communicatively coupled to the user device 102, the wearable device 106, the wearables server 108, and/or the fitness class server 110. The recommendation system 102 may be communicatively coupled to the wearable device 106, the wearables server 108, and/or the fitness class server 110 via the user device 104. The recommendation system 102 may be configured to receive data from the wearables device 106, the wearables server 108, and/or the fitness class server 110 indirectly via the user device 104. The recommendation system 102 may be configured to receive data directly from the user device 102 and/or the fitness class server 110, and indirectly from the wearables device 106 and/or the wearables server 108 (e.g. via the user device 104).

One or more of the elements of the recommendation system 102 may be configured to receive the user data from the user device 104. One or more of the elements of the recommendation system 102 may be configured to receive the fitness class data from the fitness class server 110. One or more of the elements of the recommendation system 102 may be configured to receive the fitness class data from the user device 104. One or more of the elements of the recommendation system 102 may be configured to receive the measurement data from the user device 104. One or more of the elements of the recommendation system 102 may be configured to receive the measurement data from the wearable device 106. One or more of the elements of the recommendation system 102 may be configured to receive the measurement data from the wearables server 108.

The application server 112 may be configured to receive and process the user data. The user health data and/or the user fitness data may be obtained by the application server 112 via user input at the user device 104. The user input may be in response to a prompt in an application running on the user device 104, the prompt displayed by a user interface of the user device 104. The prompt may request information that may be the basis for the user health data and/or the user fitness data. The user health data and/or the user fitness data may be obtained by the application server 112 via a measurement taken by the wearable device 106. For example, the wearable device 106 may track the user's heart rate, steps, and location. Measurement data collected by the wearable device 106 may be used to determine information about the user's exercise routine, level of health and fitness, and so forth.

The application server 112 may be configured to receive and process the fitness class data. The fitness class data may correspond to information about a fitness class. The fitness class data may be obtained from the fitness class server 110. The fitness class data may be obtained directly by the application server 112. The application server 112 may obtain the fitness class data via the user device 104. An application such as a web extractor application may run on the application server 112 (or otherwise on the health and fitness server) and may scrape the fitness class server 110 for the fitness class data. The application server 112 may preprocess the fitness class data and cause the fitness class data to be stored in the data server 114.

The data server 114 may be a knowledge database. The knowledge database may store health-related information and facts (i.e. knowledge about general health and a specific user's health). Accordingly, the data server 114 may be referred to as a health information database. The data server 114 may be in communication with the application server 112. The data server 114 may be segmented into distinct databases associated with distinct health information. For example, the data server 114 may include a user profile database and a fitness class database. The user profile database may be configured to store the user data; The fitness class database may be configured to store the fitness class data. The data server 114 may be a relational database and/or a non-relational database. For example, the user profile database may be relational, and the fitness class database may be non-relational. The user profile database and/or the fitness class database may have relational portions and non-relational portions (e.g. the database is divided into a relational database and a non-relational database, and queries are managed by an intermediary application such as the application server 112).

The expert rules database 118 may be part of the data server 114 or may be distinct from the data server 114 (e.g. may be physically or virtually remote from the data server 114). The expert rules database 118 may be part of the recommendation server 116. The expert rules database 118 may be configured to store rule data that corresponds to a rule for making a health and/or fitness recommendation. The rule may have been created by a health expert or a fitness expert. The rule may have been promulgated by an organization that sets health and/or fitness standards. The rule may be based on empirical evidence that relates the health and/or fitness recommendation positively with a particular health and/or fitness outcome.

The expert rules database 118 may include a relational database or a non-relational database. The expert rules database 118 may include a relational database that relates activity data with outcome data. The activity data may correspond to a type of activity (e.g. a physical exercise activity, a nutrition activity, a mindfulness activity, and so forth). The outcome data may correspond to physiological effects of participating in the activity. For example, the expert rules database 118 may include a relational database that includes row entries for “minutes of exercise per week” (for example) indexed to column entries for “average resting heart rate.” As another example, the expert rules database 118 may include a non-relational database that includes entries for data mined from peer-reviewed publications on health and/or fitness. Natural language processing may be implemented, such as in the recommendation engine 120, to apply rules in the non-relational database to user data or fitness class data.

The recommendation engine 120 may be programming installed on a server such as the recommendation server 116. The recommendation engine 120 may be in communication with the application server 112, the data server 114, the expert rules database 118, and/or the wearables application server 126. The recommendation server 116 may be configured to determine and/or output health objective data based on the user data. The health objective data may correspond to a preference of the user. The health objective data may correspond to a health-related goal for the user.

The health objective data may be determined directly based on the user data. For example, the user data may explicitly include a preference of the user. As another example, the user data may explicitly include a health-related goal of the user. For example, the user data may indicate the user has a preference for spin classes. The health objective data may correspond to choosing a spin class for the user. As another example, the user data may indicate the user wants to increase the user's strength. The health objective data may correspond to choosing a class where the user can increase the user's strength. The health objective data may be determined by a third party such as a doctor, a coach, and/or a trainer.

The health objective data may be determined inferentially based on the user data and/or generic data. The generic data may be stored in a database such as on the data server 115, in the expert rules database 118, and so forth. Generic may refer to information and/or data that is not specific to an individual, but rather that is shared and/or corresponds to a group of individuals (e.g. may be crowd-sourced). The generic data may include generic preference data. The generic preference data may correspond to a generic health- and/or fitness-related preference. The generic data may include generic goal data. The generic goal data may correspond to a generic health- and/or fitness-related goal.

The recommendation engine 120 may be configured to generate a tag for an element of the user data. The tag may correspond to the health information or the fitness information about the user. The recommendation engine 120 may be configured to determine the tag corresponds to the generic data. The recommendation engine 120 may be configured to determine, based on the tag corresponding to the element of the user data and the generic data, that the preference of the user is the generic health- and/or fitness-related preference. The recommendation engine 120 may be configured to determine, based on the tag corresponding to the element of the user data and the generic data, that the health-related goal of the user is the generic health- and/or fitness-related goal.

For example, the user data may indicate the user is overweight. The recommendation engine 120 may compare the user's weight to rule data in the expert rules database that corresponds to a healthy weight for individuals in the user's demographic. The recommendation engine 120 may determine the user's weight exceeds the healthy weight. The health recommendation engine 129 may be configured (i.e. through programming) to output weight loss data as the health objective data in response to the user's weight exceeding the healthy weight.

As another example, the recommendation engine 120 may determine the health objective data inferentially using natural language processing. The user data may include qualitative statements about the user's goals (e.g. “I would like to be more active”). The qualitative statements may be parsed and tagged. The tags associated with the language in the qualitative statements may be associated with tags for other user data or for rules in the expert rules database. For example, “more” directly before “active” may be tagged as “exercise frequency.” Routine data corresponding to the user's exercise routine may also be tagged with “exercise frequency.” The recommendation engine 120 may infer that the qualitative statement indicates an increase in the exercise frequency and may output the health objective data to correspond to increasing how frequently the user exercises.

The recommendation engine 120 may be configured to match an element of the health objective data with a corresponding element of the rule data in the expert rules database 118. The recommendation engine 120 may be configured to match the element of the health objective data with the element of the rule data by inferring the health objective data correspond to outcome data in the expert rules database 118. The rule data may correspond to actions and/or activities in which the user may engage. The actions and/or activities may be associated with a preference of the user. The actions and/or activities may be associated with a health and/or fitness-related goal for the user. For example, the user data may include goal data that indicates the user has set a goal to increase her takeoff speed for running. An entry in the expert rules database may indicate hip flexor exercises increase takeoff speed. The recommendation engine 120 may match the goal—increasing takeoff speed—with the rule—perform hip flexor exercises.

The recommendation engine 120 may be configured to match the element of the rule data with an element of the fitness class data. The recommendation engine 120 may be configured to match the element of the rule data with the element of the fitness class data by inferring the fitness class data corresponds to the activity data in the expert rules database 118. The fitness class data may correspond to preference factors associated with the fitness class. The fitness class data may correspond to outcomes associated with the fitness class. The recommendation engine 120 may be configured to match the element of the health objective data with the element of the rule data by inferring the health objective data corresponds to the outcomes. For example, the fitness class data may indicate that exercises performed in the fitness class increase hip flexor strength. The recommendation engine 120 may match the element of the rule data that recommends increasing hip flexor strength with the element of the fitness class data that indicates hip flexor strength is increased in the fitness class.

The recommendation engine 120 may be configured to determine recommendation data based on the element of the expert rules data matching with the element of the fitness class data. The recommendation data may correspond to a recommendation of the fitness class for the user. The recommendation server 116 may be configured to output the recommendation data to the application server 112. The application server 112 may be configured to receive the recommendation data from the recommendation server 116. The application server may be configured to convert the recommendation data from a raw format output by the recommendation server 116 to a user-readable format. The recommendation system 102 may be configured to output to the user device 104 via the application server 112 the recommendation data in the user-readable format. The recommendation system 102 may be configured to output the recommendation data in the user-readable format to the wearable device 106. The recommendation system 102 may be configured to output the recommendation data in the user-readable format to the wearables server 108.

The recommendation of the fitness class may be based at least in part on the user health data or the user fitness data. The recommendation of the fitness class may be based at least in part on the fitness class data. The recommendation data may be based on the element of the rule data matching with the element of the fitness class data, where the element of the rule data is matched to health objective data associated with the user. The fitness class may be recommended for the user based on a goal of the user reflected in the user data.

The recommendation of the fitness class for the user may be based at least in part on the location of the user relative to the location of the fitness class. For example, the recommendation of the fitness class for the user may be based in part on an actual distance between the location of the user and the location of the fitness class being less than the maximum distance indicated in the location range data for the user. The recommendation engine 120 may be configured to correlate the user location data with the fitness class data, and the recommendation data may be based at least in part on the user location data correlating with the fitness class data.

The wearables server 108 may directly communicate with the wearable device 106. For example, the wearable device and the wearables server 108 may be connected via the Internet. The wearables server 108 may be configured to receive sensor data from the wearable device 106. The sensor data may correspond to a measurement taken by the wearable device 106 such as a physiological measurement of a physiological characteristic of the user. The wearables server 108 may be configured to output, to the user device 104, measurement data that corresponds to the sensor data. The measurement data may be incorporated into the user data (e.g. the user health data or the user fitness data). The application server 112 may receive the measurement data via the user device 104.

The sensor data may correspond to a biometric reading taken by the wearable device 106 (e.g. a heart rate of the user). The sensor data may correspond to an activity reading taken by the wearable device 106 (e.g. a number of steps taken by the user in a given time period). The recommendation of the fitness class for the user may be based in part on the biometric reading or the activity reading.

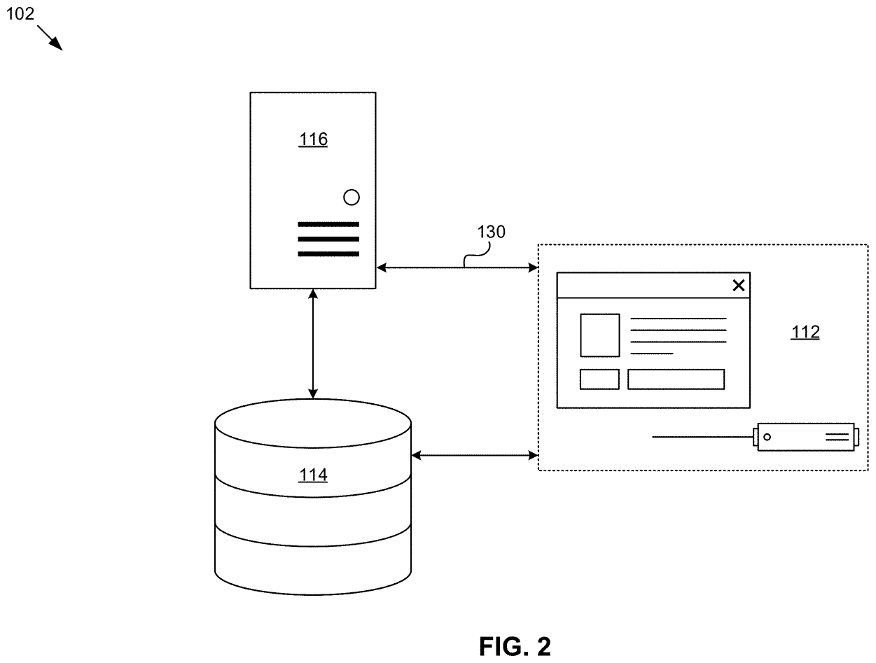

FIG. 2 illustrates the recommendation system 102, according to an embodiment. The recommendation system 120 may include three components: an application server (e.g. the application server 112), a health information database (e.g. the data server 114), and a health recommendation server (e.g. the recommendation server 116). The application server and the recommendation server may be segregated such that bandwidth requirements on one server do not affect bandwidth availability on the other server. By keeping the servers segregated, server calls to the application server from client devices may be handled without delay or lag due to processing occurring at the health recommendation server.

The data server 114 may be communicatively coupled via the communication links 130 to the application server 112 and/or the recommendation server 116. The data server 114 may include a user profile database configured to store the user data, a fitness class database configured to store the fitness class data, and an expert rules database configured to store rule data that corresponds to a rule for making a health recommendation.

The recommendation server 116 may be configured to output health objective data based on the user data. The health objective data may be determined directly and/or inferentially based on the user data. The recommendation server 116 may be configured to match an element of the health objective data with a corresponding element of the rule data. The rule data may correspond to actions by the user associated with the health objective data. The recommendation server 116 may be configured to match the element of the rule data with an element of the fitness class data. The element of the fitness class data may correspond to an aspect of the fitness class that aligns with the health objective data based on the rule data. The recommendation server 116 may be configured to output recommendation data that corresponds to a recommendation of the fitness class for the user.

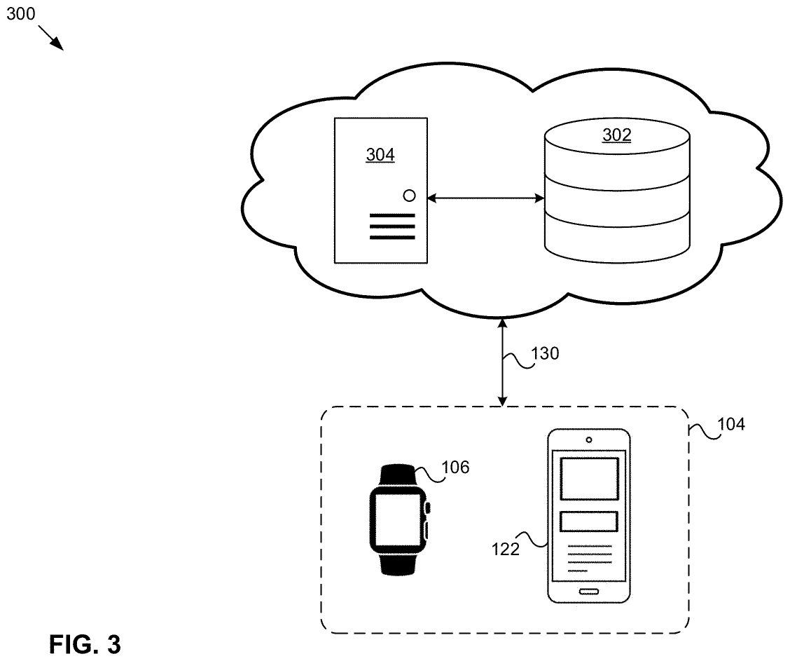

FIG. 3 illustrates a cloud-based recommendation system 300, according to an embodiment. Having the recommendation system based in the cloud may enable a user to receive recommendations when the user is on-the-go and/or traveling. For example, the user may travel away from the user's home, such as to another city and/or state. The user may participate in a fitness class near the user's home. The user may wish to participate in the fitness class while the user is away from home. The user may utilize the cloud-based recommendation system 300 to identify times and locations for similar fitness classes while the user is away from home.

The cloud-based recommendation system 300 may include a database server 302 and a processing server 304. The database server 302 and/or the processing server may be communicatively coupled to the user device 104 (e.g. the smartphone 122 and/or the wearable device 106) via the communication link 130.

The database server 302 may include one or more distinct databases. The distinct databases may store different types of information. For example, the database server 302 may include a user knowledge database, a fitness knowledge database, and/or an expert rules database. The distinct databases may store information in different ways, such as relationally or non-relationally. The processing server 304 may include one or more distinct servers tasked for different purposes. For example, the processing server 304 may include an application server, a data server, a website server, an artificial intelligence server, and so forth.

The fitness knowledge database may store fitness class data that corresponds to information about the fitness class. The fitness knowledge database may be a relational database or a non-relational database. The user knowledge database may store health and fitness data that corresponds to health and fitness information about a user. The user knowledge database may be a relational database or a non-relational database. The expert rules database may store expert rules data that corresponds to rules for making health and fitness recommendations. The rules may be based on information from expert health and/or fitness resources. The expert rules database may be a relational database or a non-relational database.

The processing server 304 and the database server 302 may be communicatively coupled. The processing server may include a fitness inference engine configured to access data stored on the database server 302 and make recommendations based on the data. The fitness inference engine may be configured to determine whether a difference between the fitness class location and the user location is within a range specified by the user. The fitness inference engine may be configured to output touchpoint data. An individual touchpoint may include a threshold level of commonality or a threshold degree of alignment. The touchpoints may be determined between the fitness class data and the user data, between fitness outcome data associated with the fitness class and the user data, or between the fitness class data, the fitness outcome data, and the user data.

The fitness inference engine may be configured to determine and/or output recommendation data that recommends the fitness class for the user. The recommendation may be generated in response to the difference between the class location and the user location being the range specified by the user. The recommendation may be generated in response to the touchpoint data including a threshold quantity of individual touchpoints that satisfy the threshold level of commonality or the threshold degree of alignment. The threshold quantity may correspond to a number of touchpoints, such as one touchpoint, two touchpoints, three touchpoints, and so forth. The threshold quantity may correspond to having a minimum number of touchpoints from different categories. For example, the threshold quantity may correspond to having touchpoints in a location category and an exercise type category.

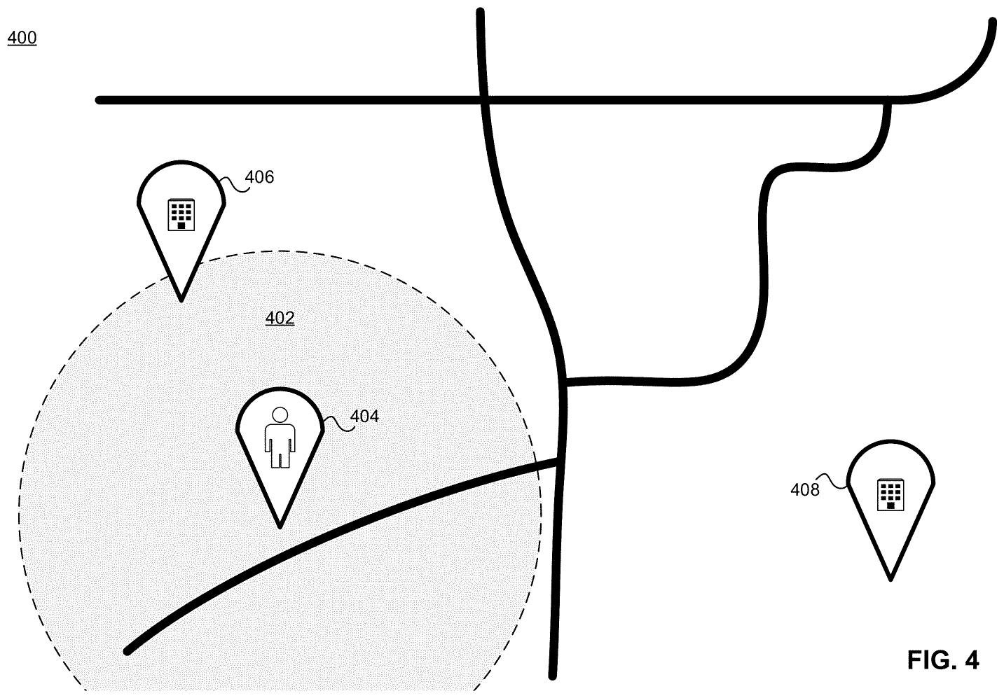

FIG. 4 illustrates a map 400 displaying geolocation for the user and the fitness class, according to an embodiment. The user may not know where fitness classes are that the user would like to participate in. The recommendation system 102 may recommend fitness classes to the user based on which fitness classes fall within a specified range 402 of the user's location 404. For example, the user may be making new efforts to get in shape and may wish to know what fitness classes are near the user's home. The user may set a range from the user's home and the recommendation system 102 may recommend classes within that range.

The map 400 may show the user's location 404, the range 402 from the user's location, a first fitness class location 406, and a second fitness class location 408. The range 402 may be selected by the user and may be reflected in range data as part of the user data. The first fitness class location 406 may fall within the range 402. The second fitness class location 408 may fall outside the range 402. The recommendation system 102 may be configured to recommend a fitness class at the first fitness class location 406 in response to the first fitness class location 406 falling within the range 402.

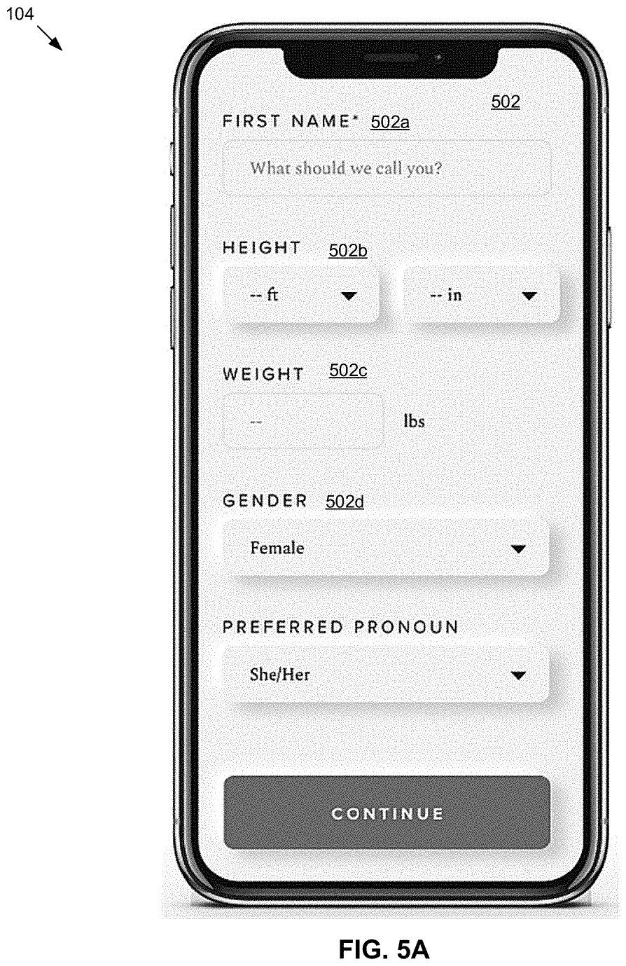

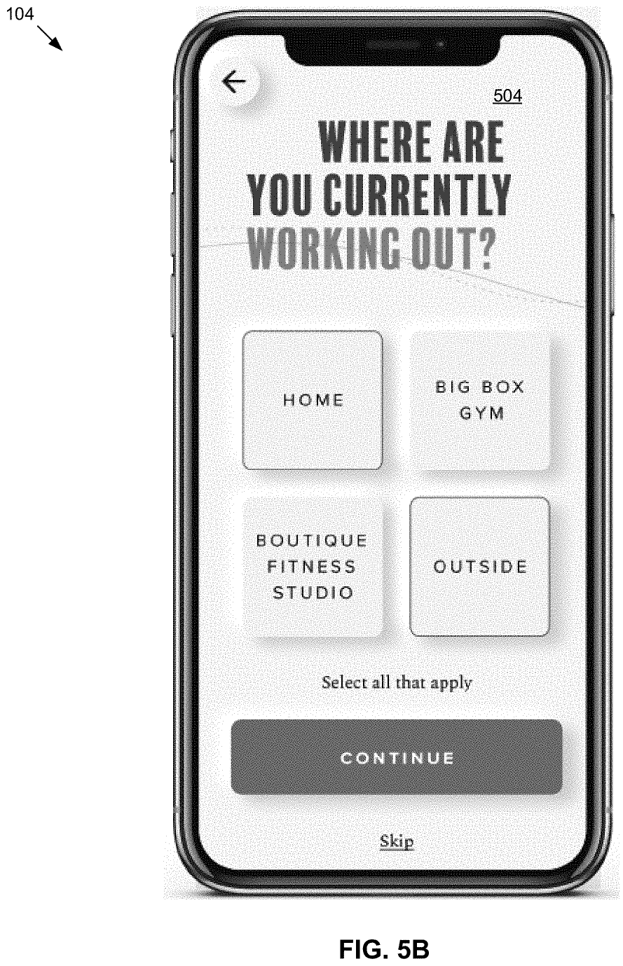

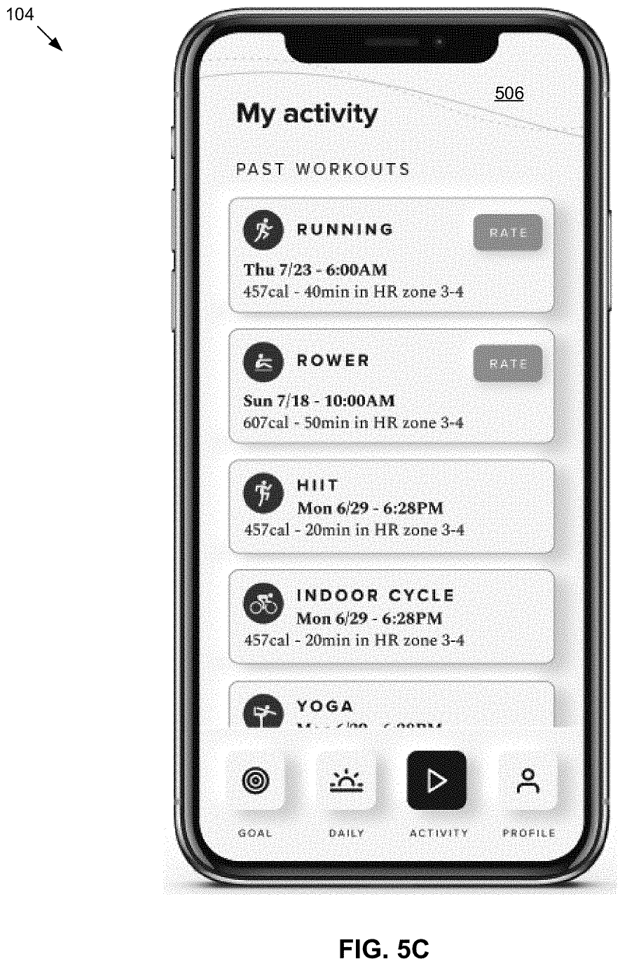

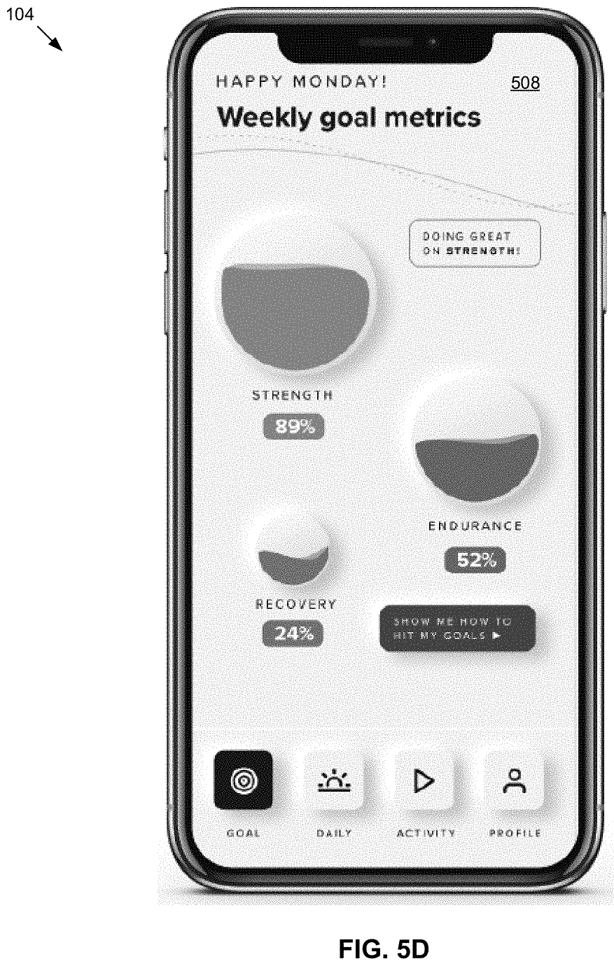

FIG. 5A illustrates the user device 104 displaying a user interface 502 where a user can input information about the user, according to an embodiment. The recommendation system 102 may recommend a specific fitness class for the user based on the user's personal information. The user's personal information may include demographic information such as the user's name 502a, height 502b, weight 502c, gender 502d, and so forth. The recommendation system 102 may include a client application installed on the user device 104. The client application may collect the user's information via input by the user on the user device 104.

FIG. 5B illustrates the user device 104 displaying a user interface 504 where the user can input fitness information about the user, according to an embodiment. The recommendation system 102 may recommend a specific fitness class for the user based on the user's current exercise routine. The user's exercise routine may include when the user exercises, where the user exercises, and what types of activities the user engages in. The user interface 504 may prompt the user to input information about the user's exercise routine. The input may be stored with the user data and used by the recommendation system 102. For example, the setting in which the user typically exercises may be at a gym, at home, outdoors, and so forth. The fitness class data may include class setting data that corresponds to a setting in which the fitness class is held. The class may be held indoors, outdoors, or online. The recommendation system 102 may recommend a fitness class that matches the setting where the user typically exercises.

FIG. 5C illustrates the user device 104 displaying fitness information about the user on the user interface 506, according to an embodiment. The fitness information may include information about classes or exercises the user has engaged in. The recommendation system 102 may use this information in recommending future classes and/or exercises for the user. The recommendation system 102 may update recommendations for the user as the user inputs routine information into the user device 104. The updated information may be included with the user data and the recommendation system 102 may update the recommendations for the user. This may allow the user and/or the recommendation system 102 to track the user's fitness progress.

FIG. 5D illustrates the user device 104 displaying information about a goal of the user on a user interface 508, according to an embodiment. The recommendation system 102 may enable the user to track progress towards the user's goal and/or update recommendations based on the user's progress. The user may select which goals the user would like to achieve or the recommendation system 102 may recommend goals for the user based on the user data. By having a visual indication of the user's progress, the user may be more motivated to continue with the recommendations output by the recommendation system 102.

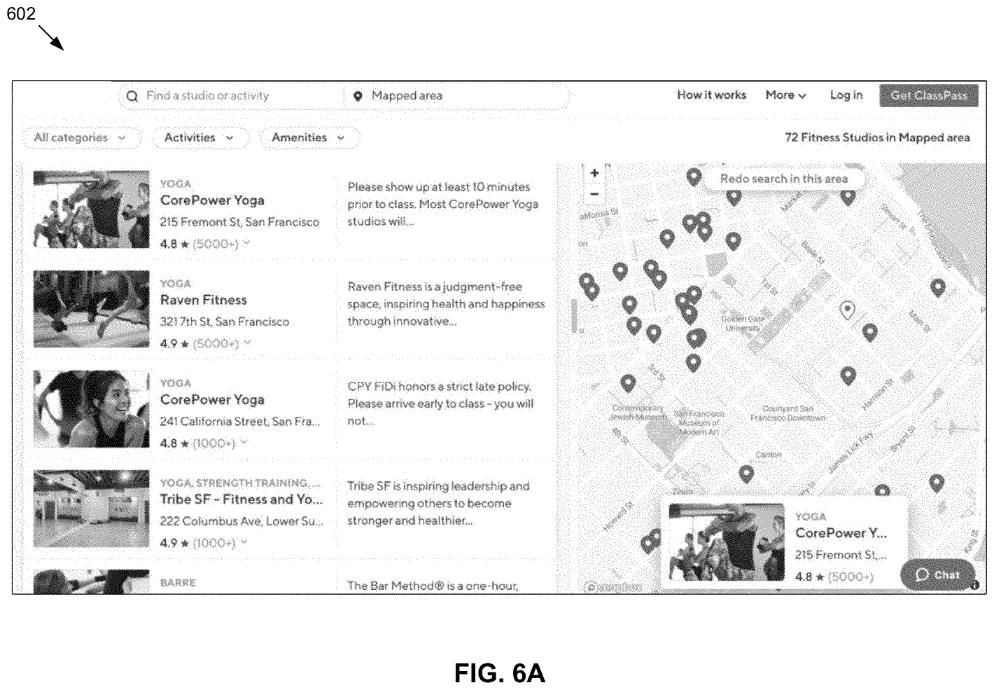

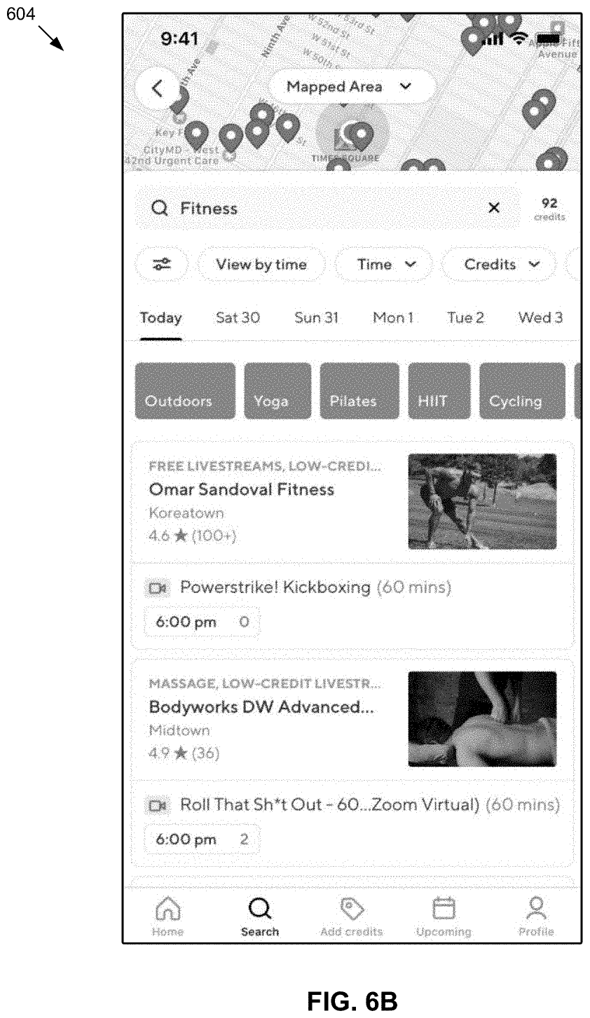

FIG. 6A illustrates a website 602 where the recommendation system 102 may source fitness class data, according to an embodiment. By sourcing fitness class data from third-party resources, the recommendation system 102 may reduce an amount of storage required by the system. Additionally, by sourcing fitness class data from third-party resources, the recommendation system 102 may remain up-to-date as fitness class information changes. For example, a particular fitness class may change days or change time. The recommendation system 102 may obtain the updated information in real-time and thereby keep the user updated with the most recent information for the fitness class.

The recommendation system 102 may include a data extraction application. The data extraction application may be installed on, for example, the application server 114 and/or the recommendation server 116. The recommendation system 102 may include a separate server dedicated to data extraction. The data extraction application may be configured to retrieve the fitness class data from the third-party data resource.

FIG. 6B illustrates a mobile device application 604 where the recommendation system 102 may source fitness class data, according to an embodiment. The mobile device application 604 may be associated with an account owned by the user for a fitness class information resource such as ClassPass™ or MINDBODY™. The recommendation system 102 may access the fitness class information in the mobile device application 604 using the user's credentials. The recommendation system 102 may protect the user's privacy by not storing the user's credentials and instead accessing the fitness class information in the mobile device application 604 through the user device 104.

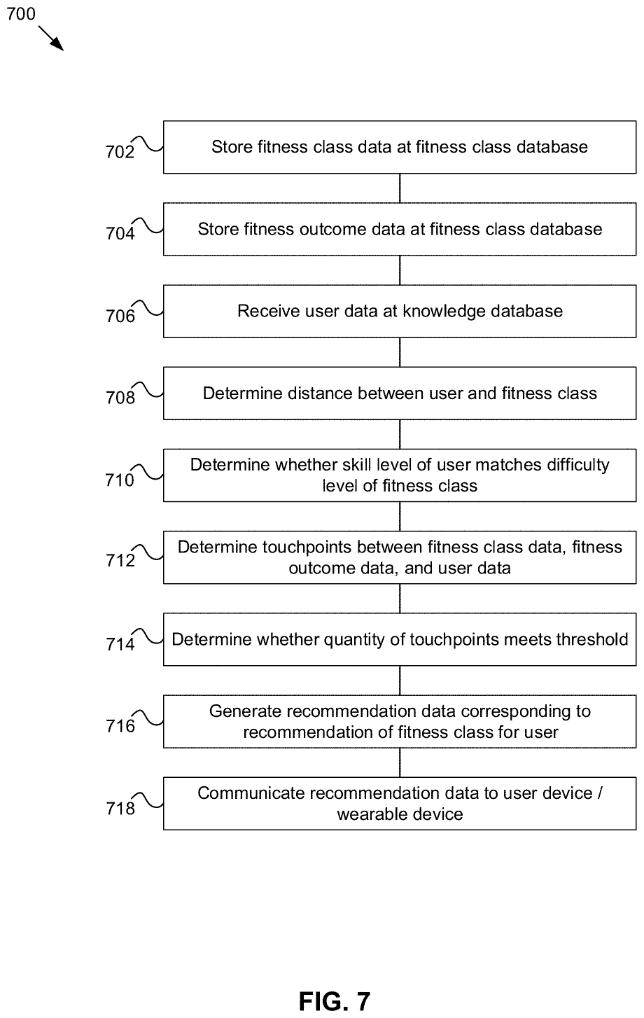

FIGS. 7-14 illustrate various methods for making health and/or fitness recommendations. The methods may be executed using one or more of the devices described herein, such as the health and fitness device network 100, the recommendation system 102, the user device 104, the wearable device 106, the wearables server 108, the fitness class server 110, the application server 112, the data server 114, the recommendation server 116, the expert rules database 118, and/or the recommendation engine 120. As used herein, fitness inference engine may refer to the recommendation engine 120 and/or functionality of the recommendation engine 120.

FIG. 7 illustrates a method 700 of generating a fitness class recommendation for a user, according to an embodiment. A user may have health and/or fitness goals but may not know how to accomplish those goals. The user may not know what goals to set but may generally want to increase his level of health and/or fitness. The method 700, when implemented by a system such as the recommendation system 102, may enable the user to identify specific goals and/or specifically how to accomplish those goals by recommending specific activities and/or classes. The specificity may be highly granular, including specific days, times, and/or locations for the activities and/or classes.

The method 700 may include storing fitness class data (block 702). The fitness class data may be stored, for example, in a fitness knowledge database. The fitness class data may include class location data that corresponds to a location of a fitness class. The fitness class data may include schedule data that corresponds to a schedule for the fitness class. The fitness class data may include instructor data that corresponds to information about an instructor of the fitness class. The fitness class data may include class exercise data that corresponds to a type of exercise performed in the fitness class. The fitness class data may include difficulty data that corresponds to a difficulty level of the fitness class.

The method 700 may include storing fitness outcome data (block 704). The fitness outcome data may be stored, for example, in the fitness knowledge database. The fitness outcome data may correspond to a fitness outcome associated with a particular type of exercise. The fitness outcome data may correspond to a fitness outcome associated with a particular pattern of activity, such as alternating between strength training and endurance training.

The method 700 may include receiving user data (block 706). The user data may be received at, for example, a user knowledge database. The user data may be received from a user device. The user data may include user location data that corresponds to a location of a user. The user data may include fitness routine data that corresponds to a fitness routine of the user. The user data may include biometry data that corresponds to biometric information about the user. The user data may include goal data that corresponds to a fitness goal for the user. The user data may include preference data that corresponds to a fitness class preference of the user. The user data may include and skill data that corresponds to a skill level of the user in a particular fitness class. The skill level may be self-identified. The skill level may be based on an objective standard and/or certification.

The method 700 may include determining whether a difference between the class location data and the user location data is reflected in location range data (block 708). The determination may be made by a fitness inference engine in communication with the fitness knowledge database and the user knowledge database. The location range data may correspond to a maximum distance preferred by the user between the location of the user and the location of the fitness class. The location range data may be stored in the fitness knowledge database. The location range data may be received from the user device.

The method 700 may include determining whether a difference between the difficulty data and the skill data is reflected in skill range data (block 710). The skill range data may correspond to a maximum difference between the difficulty level of the fitness class and the skill level of the user. The skill range data may be stored in the fitness knowledge database. The skill range data may be received from the user device.

The method 700 may include determining, by the fitness inference engine, touchpoint data (block 712). The touchpoint data may correspond to a set of touchpoints between the fitness class data, the fitness outcome data, and/or the user data. An individual touchpoint in the set of touchpoints may include a threshold level of commonality between the fitness routine data and the class exercise data. An individual touchpoint in the set of touchpoints may include a threshold level of commonality between the fitness routine data and the schedule data. An individual touchpoint in the set of touchpoints may include a threshold level of commonality between the goal data and the fitness outcome data. An individual touchpoint in the set of touchpoints may include a threshold degree of alignment of the biometry data with the difficulty data. An individual touchpoint in the set of touchpoints may include a threshold degree of alignment of the preference data with the class exercise data. An individual touchpoint in the set of touchpoints may include a threshold degree of alignment of the preference data with the instructor data.

The method 700 may include determining whether a threshold quantity for the set of touchpoints satisfies the threshold level of commonality or the threshold degree of alignment (block 714). The determination may be made by the fitness inference engine.

The threshold level of commonality between the fitness routine data and the class exercise data may include the user exercise data matching the class exercise data. The threshold level of commonality between the goal data and the fitness outcome data may include the goal data matching the fitness outcome data. The threshold level of commonality between the goal data and the fitness outcome data may include the fitness outcome leading to the fitness goal. The fitness outcome may correspond to the fitness outcome data and the fitness goal may correspond to the goal data.

The threshold level of commonality between the fitness routine data and the schedule data may be satisfied when at least part of the class day data is the same as at least part of the routine day data. As another example, the threshold level of commonality between the fitness routine data and the schedule data may be satisfied when the start time data is reflected in the time of day data; or the schedule data may include the first null data.

The threshold degree of alignment of the preference data with the instructor data may be satisfied when gender data for the instructor is the same as the preference data for the gender preferred by the user. The threshold degree of alignment of the preference data with the instructor data may be satisfied when a difference between rating data for the instructor and the rating preference data is reflected in rating range data. The rating range data may be a minimum threshold (e.g. the user prefers an instructor and/or class with a rating of at least 4). The rating range data may correspond to a maximum rating difference (e.g. the user prefers instructors and/or classes with a rating of 4, and the range allows for instructors and/or classes with a rating of at least 3.5). This may be useful when an instructor and/or class with a minimum rating preferred by the user is not available based on other preferences and/or recommendations for the user. The rating range data may be stored in the fitness knowledge database and/or may be received from the user device.

The threshold degree of alignment of the preference data with the instructor data may be satisfied when a difference between qualification data for the instructor and the qualification preference data is reflected in qualification range data. The qualification preference data may correspond to a preferred qualification of the instructor and/or the class by the user. The qualification range data may correspond to a range of qualifications that satisfy the user's preference (e.g. a maximum difference between what the user prefers and what the user will accept or a maximum difference between what the user prefers and what is recommendable and/or available). For example, the user may prefer top qualifications, but such options may not be available within the user's location range. The qualification range data may be stored in the fitness knowledge database and/or may be received from the user device.

The method 700 may include generating recommendation data (block 716). The recommendation data may be generated by the fitness inference engine. The recommendation data may correspond to a recommendation of a particular fitness class, fitness activity, and/or exercise for the user. The recommendation data may be generated in response to the difference between the class location data and the user location data being reflected in the location range data. The recommendation data may be generated in response to the difference between the difficulty data and the skill data being reflected in the skill range data. The recommendation data may be generated in response to the threshold quantity for the set of touchpoints satisfying the threshold level of commonality or the threshold degree of alignment.

The class location data may include class geographic data that indicates a geographic location of the class (e.g. a physical address). The class location data may include digital address data that indicates the fitness class is a virtual class on a digital platform. The digital platform may, for example, be the fitness class server 110. The user location data may include user geographic data that indicates a physical location of the user (e.g. the user's physical address). The user location data may include region data for a geographic region associated with the user. The region data may be the same as or similar to the range data for the user. The user location data may include travel data that corresponds to a travel routine of the user (e.g. the user's daily commute for work, and so forth).

The difference between the class location data and the user location data may be reflected in the location range data when a difference between the geographic data for the class and the user geographic data is directly reflected in the location range data (e.g. within 5 blocks, within 1 mile, within 5 miles, within 10 miles, and so forth). The difference between the class location data and the user location data may be reflected in the location range data when the class geographic data is reflected in the region data for the user (e.g. the address of the class is in the same city as the user). The difference between the class location data and the user location data may be reflected in the location range data when the class geographic data is reflected in the travel data (e.g. the class is located on the user's travel route to work). The difference between the class location data and the user location data may be reflected in the location range data when the class location data includes the digital address data (i.e. the class can be taken online at any physical location, so it is always within the range set by the user).

The method 700 may include communicating the recommendation data to a device of the user (block 718). The device may be a user device such as a smartphone or a wearable health monitoring device. The device may be a personal computer and the recommendation data may be communicated to a web browser operating on the personal computer.

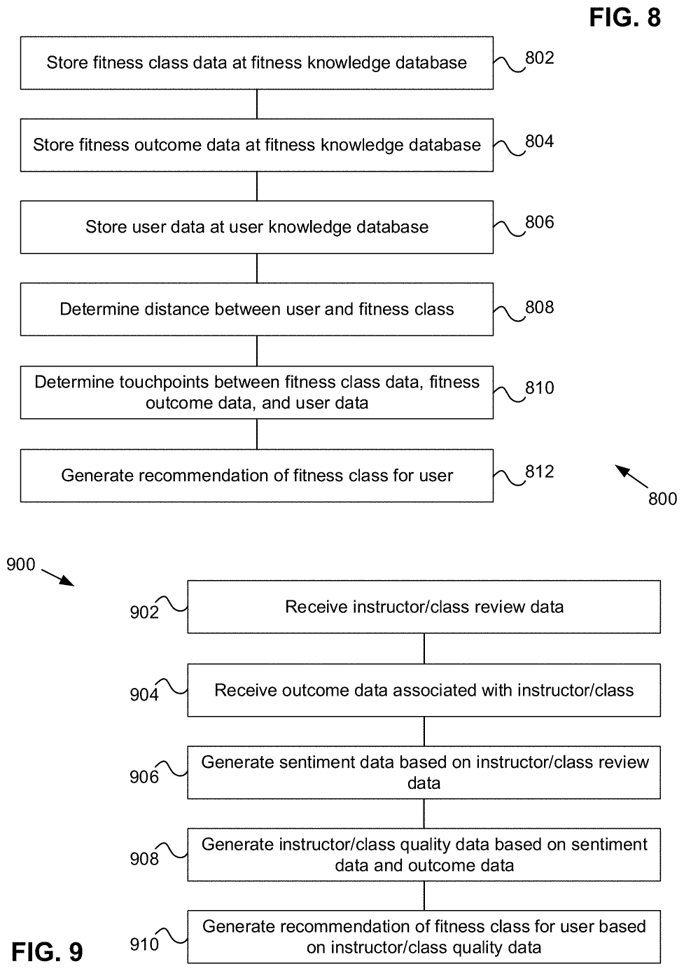

FIG. 8 illustrates another method 800 of generating the fitness class recommendation, according to an embodiment. Various data resources may be centralized in the recommendation system, enabling precise recommendation of health and fitness activities for the user. By compiling a vast amount of data on expert recommendations, fitness class and activity availability, and specific information about the user, recommendations can be tailored to the user automatically, on-the-fly, and dynamically.

The method 800 may include storing fitness class data (block 802). The fitness class data may be stored in a knowledge database. The method 800 may include storing fitness outcome data corresponding to a fitness outcome associated with the fitness class (block 804). The fitness outcome data may be stored in the knowledge database. The method 800 may include storing user data (block 806). The user data may be stored in a user database or the knowledge database. The user data may be received from a user device.

The method 800 may include determining whether a difference between the class location and the user location is within a specified range (block 808). The range may be specified by the user or may be inferred based on the user's location and/or travel routine. The determination may be made using class location data, user location data, and/or location range data. The determination may be made by an inference engine in communication with the knowledge database and/or the user database.

The method 800 may include determining touchpoint data (block 810). The touchpoint data may be determined similar to that described regarding the method 700 depicted in FIG. 7. The method 800 may include communicating the recommendation data to a device of the user (block 812).

FIG. 9 illustrates a method 900 of using instructor and/or class rating data to recommend the fitness class, according to an embodiment. The user may wish to take a class that is effective and/or enjoyable. The user may wish to take a class from an instructor that is effective and/or well-liked by attendees of the class. However, a well-liked class and/or instructor is not always effective, and effective classes and/or instructors are not always well-liked. The method 900 may enable objective recommendation of fitness classes and instructors that will be effective and enjoyable for the user based on the user's specific preferences and/or goals.

The method 900 may be implemented in conjunction with various other methods described herein. The method 900 may include receiving review language data (block 902). The review language data may correspond to reviews of the instructor by attendees of the fitness class taught by the instructor. The review language data may correspond to reviews of the fitness class by attendees of the fitness class. The method 900 may include receiving outcome data associate with the instructor and/or class (block 904). The outcome data may be part of the fitness class data that is retrieved by the recommendation system from the third-party resource. The outcome data may be indicated in the natural language data. The outcome data may correspond to outcomes (e.g. losing weight, increasing speed, and so forth) associated with the instructor and/or the fitness class.

The method 900 may include generating sentiment data based on the review language data (block 906). The sentiment data may correspond to a sentiment for the instructor based on the natural language reviews. The sentiment data may correspond to a sentiment for the fitness class based on the natural language reviews. For example, the natural language reviews may indicate the instructor is friendly. The sentiment data may accordingly reflect a positive sentiment for the instructor. As another example, the natural language reviews may indicate the fitness class is challenging and rewarding. The sentiment data may accordingly reflect a positive sentiment for the fitness class. The sentiment data may be a rating for the instructor and/or the class. The sentiment data may be rating data. The rating data may be a numerical value that corresponds to whether or not the instructor or class is recommendable. For example, the rating data may include the number 10, which may correspond to the instructor and/or class being highly recommendable.

The method 900 may include generating overall quality data for the instructor and/or the class based on the sentiment data and the outcome data (block 908). A first percentage of the quality data may be based on the rating data. The first percentage may be in a range from ten percent to ninety percent. The first percentage may be in a range from thirty percent to fifty percent. The first percentage may be approximately forty percent. A remaining percentage of the quality data is based on the outcome data. As an example, forty percent of the quality data may correspond to the rating data and sixty percent of the quality data may correspond to the outcome data. The quality data may be represented by a numerical value. The numerical value may be a sum of the rating data weighted to forty percent and the outcome data weighted to sixty percent.

The method 900 may include generating recommendation data for the instructor and/or the class based on the quality data (block 910). For example, the quality data may indicate the instructor and/or class is effective and enjoyable, making the fitness class taught by the instructor highly recommendable. A rule for recommending a fitness class may be that highly recommendable instructors and/or classes are recommended. In response to the quality data indicating the instructor and/or the class is highly recommendable, the fitness class may be recommended. Quality data for the instructor may be combined with quality data for the class to determine an overall recommendation for the fitness class. For example, the instructor may be highly recommendable, and the class may be moderately recommendable. An overall recommendation for the fitness class may be that it is moderately to highly recommendable.

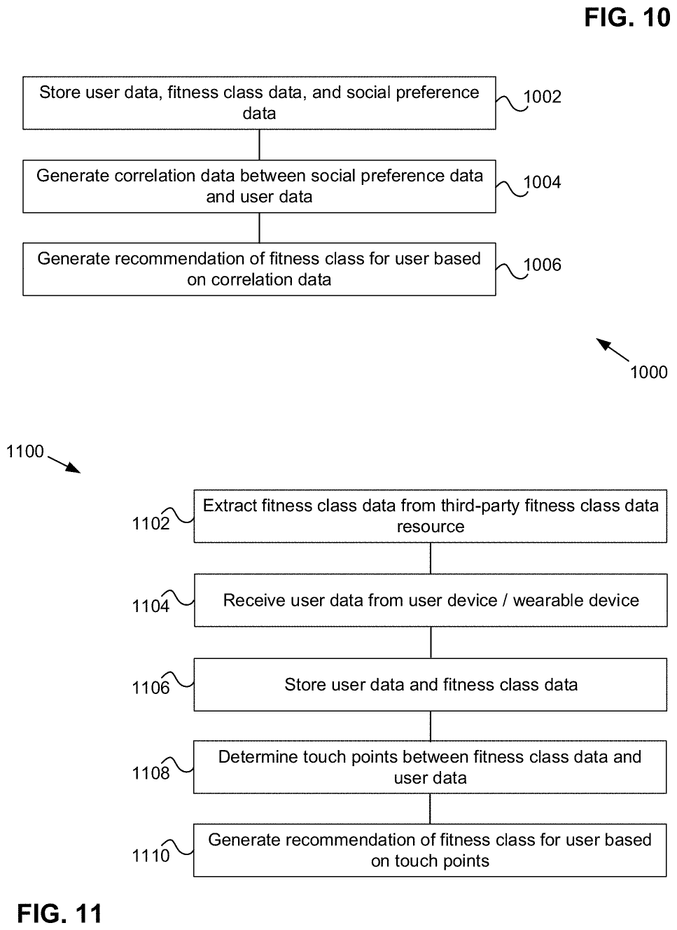

FIG. 10 illustrates a method 1000 of using a correlative filter to recommend the fitness class, according to an embodiment. The user may wish to take a class that is effective and/or enjoyable. The user may wish to take a class from an instructor that is effective and/or well-liked by attendees of the class. However, a well-liked class and/or instructor is not always effective, and effective classes and/or instructors are not always well-liked. The method 1000 may enable objective recommendation of fitness classes and instructors that will be effective and enjoyable for the user based on the user's specific preferences and/or goals.

The method 1000 may include storing user data, fitness class data, and social preference data (block 1002). The social preference data may include group demographic data that corresponds to demographic information about the group of individuals that take the fitness class. The fitness class data may include group fitness data that corresponds to fitness information about the group of individuals. The user data may include user demographic data that corresponds to demographic information about the user.

The method 1000 may include generating correlation data (block 1004). The correlation data may be generated by a collaborative filtering engine in communication with a knowledge database that stores the user data, the fitness class data, and the social preference data. The collaborative filtering engine may, for example, be implemented on the recommendation server 116. The correlation data may correspond to a correlation between the social preference data and the user data; The correlation data may correspond to a correlation between the group demographic data and the user demographic data. The correlation data may correspond to a correlation between the group fitness data and the user fitness routine data. and outputting, by the inference engine, the recommendation of the fitness class for the user based on the correlation data. The method 1000 may include generating a recommendation of the fitness class for the user based on the correlation data (block 1006).

The correlation data may be based on user preference data such as gender preference data, rating preference data, and/or qualification data. The gender preference data may correspond to a preference of the user for a specific gender of the instructor. The rating preference data may correspond to a preference of the user for a specific rating of the instructor or the class. The rating preference may be for the quality of the instructor and/or class, the sentiment of the instructor and/or the class, and/or the outcomes associated with the instructor and/or the class. The qualification data may correspond to a qualification for the instructor and/or the class preferred by the user.

FIG. 11 illustrates a method 1100 of recommending the fitness class based on data obtained from a third-party fitness class data resource, according to an embodiment. The recommendation systems described herein may include data extraction applications that crawl external data resources for information about fitness classes. This may enable the recommendation system the make recommendations based on current information.

The method 1100 may include extracting fitness class data from a third-party fitness class data resource (block 1102). The data may be extracted using a web data extraction tool such as a web crawler or a web scraper. The fitness class data may include a schedule for the class, ratings for the class and/or instructors, and/or outcomes for participants in the class. The method 1100 may include receiving user data from the user device and/or the wearable device (block 1104). The method 1100 may include storing the user data and the fitness class data (block 1106). The data may be stored, for example, in a central database. the method 1100 may include determining touchpoints between the fitness class data and the user data (block 1108). The method 1100 may include generating recommendation data that corresponds to a recommendation of the fitness class for the user (block 1110). The recommendation may be based on the touchpoints between the fitness class data and the user data.

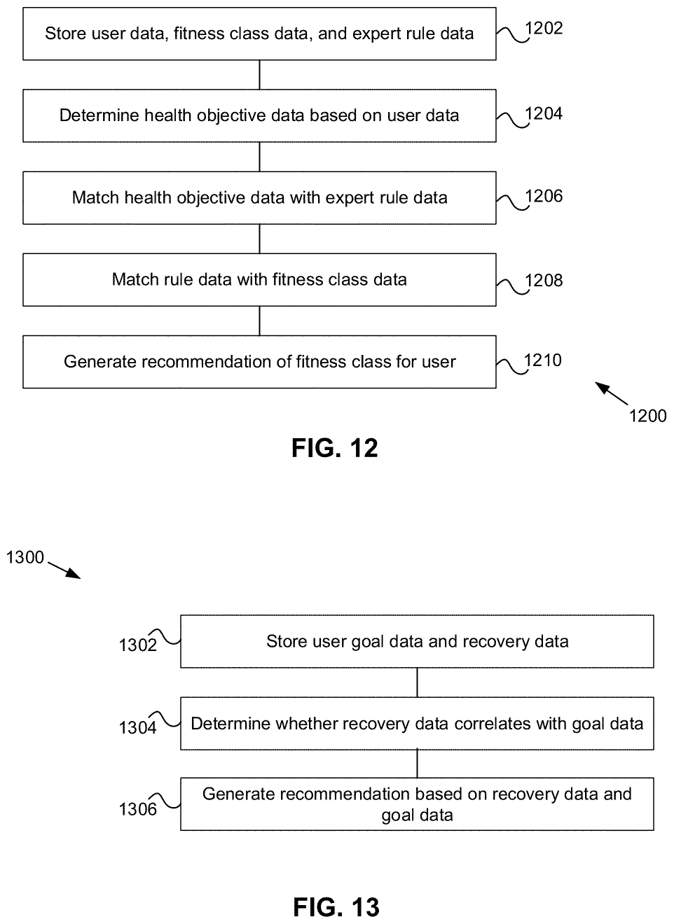

FIG. 12 illustrates a method 1200 of recommending the fitness class by matching the fitness class data and the user data to corresponding rule data, according to an embodiment. Expert insight into certain health and fitness activities may indicate certain outcomes associated with those activities. Those insights may be translated into rules for recommending those activities based on the user's health and fitness goals.

The method 1200 may include storing, in a database, user data, fitness class data, and expert rule data (block 1202). The expert rule data may include fitness outcome data that corresponds to outcomes associated with various fitness activities. The fitness outcome data may include biometry change data that corresponds to a change in biometry of participants that engage in exercises performed in the fitness class. The fitness outcome data may include vitals change data that corresponds to a change in a vital characteristic of the participants that engage in the exercises performed in the fitness class.

The biometry change data may include HRV change data. The HRV change data may correspond to a change in an HRV of the participants in the fitness class. The biometry change data may include resting heart rate change data. The resting heart rate change data may correspond to a change in a resting heart rate of the participants in the fitness class. The biometry change data may include VO₂ max change data. The VO₂ max change data may correspond to a change in a VO₂ max for the participants in the class.

The vitals change data may include weight change data. The weight change data may correspond to a change in a weight of the participants in the fitness class (e.g. participants in the fitness class lose weight). The vitals change data may include health disorder change data. The health disorder change data correspond may correspond to a change in a health disorder of the participants in the fitness class. The health disorder change data may correspond to a quality of management of a health disorder by participants in the fitness class. The quality of management may correspond to participation in the fitness class. For example, participants that previously had high blood pressure may see a reduction in blood pressure correlating with participation in the fitness class.

The method 1200 may include determining health objective data for the user based on the user data (block 1204). The health objective data may be determined directly based on the user data or inferentially based on the user data. The method 1200 may include matching an element of the health objective data with an element of the expert rule data (block 1206). For example, the user data may indicate the user is overweight. The health objective data may indicate weight loss as a goal for the user. The health objective data may be matched to expert rule data that corresponds to weight loss. The method 1200 may include matching the element of the expert rule data with an element of the fitness class data (block 1208). For example, the fitness class data may indicate a certain activity is performed in the fitness class. The expert rule data may indicate the activity corresponds with a particular outcome. The method 1200 may include generating recommendation data (block 1210). The recommendation data may correspond to a recommendation of the fitness class for the user.

FIG. 13 illustrates a method 1300 of recommending a recovery period, according to an embodiment. Resting may be as important to health and fitness as activity. A user may have a certain goal and may be engaging in the appropriate activities to achieve that goal. However, the user may be unaware that the user's routine does not include enough recovery time for the activities to be effective in helping the user accomplish the goal. The recommendation systems described herein may account for recovery in recommending fitness activities and classes to the user.

The method 1300 may include storing user goal data and recovery data, such as at a knowledge database (block 1302). The recovery data may correspond to an amount of recovery associated with a health and fitness outcome. The goal data may correspond to the health and fitness outcome. The method 1300 may include determining, such as by an inference engine, whether the recovery data correlates with the goal data (block 1304). For example, the goal data may indicate the user wishes to increase strength. The recovery data may indicate that an amount of time, such as one to three days, may be optimal for increasing strength when doing strength training exercises. The method 1300 may include generating, such as by the inference engine, recovery recommendation data in response to the recovery data correlating with the goal data (block 1306). The recovery recommendation data may be incorporated with the recommendation data that recommends the fitness class for the user.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods, and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions, and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples. The Applicant(s) reserves the right to submit claims directed to combinations and sub-combinations of the disclosed examples that are believed to be novel and non-obvious. Examples embodied in other combinations and sub-combinations of features, functions, elements, and/or properties may be claimed through amendment of those claims or presentation of new claims in the present application or in a related application. Such amended or new claims, whether they are directed to the same example or a different example and whether they are different, broader, narrower, or equal in scope to the original claims, are to be considered within the subject matter of the examples described herein.

1. A system, comprising:
a wearable device configured to:
take a measurement from a user; and
generate measurement data based on the measurement taken from the user;
a wearables server communicatively coupled to the wearable device, the wearables server configured to:
receive the measurement data from the wearable device, wherein the wearable device is configured to communicate the measurement data to the wearables server; and
communicate the measurement data to a first receiving device;
a user device communicatively coupled to the wearable device and the wearables server, the user device is configured to:
receive the measurement data from the wearable device or the wearables server;
receive an input from a user;
generate user data based on the measurement data or the input from the user, wherein the user data comprises:
user health data that corresponds to health information about a user; or
user fitness data that corresponds to fitness information about the user; and
communicate the user data to the first receiving device or a second receiving device;
a fitness class server communicatively coupled to the user device, the fitness class server configured to:
store fitness class data, the fitness class data corresponding to information about a fitness class; and
communicate the fitness class data to the user device; and
a recommendation server communicatively coupled to the user device, the recommendation server configured to:
store expert rules data that corresponds to a rule for making a health recommendation or a fitness recommendation, the rule created by a health expert or a fitness expert;
receive the user data from the user device;
receive the fitness class data from the fitness class server via the user device;
determine health objective based on the user data, wherein:
the health objective data corresponds to:
a preference of the user; or
a health-related goal for the user; and
the health objective data is determined:
directly based on the user data; or
inferentially based on the user data;
match an element of the health objective data with a corresponding element of the expert rules data, wherein the expert rules data corresponds to an activity for the user associated with the preference of the user or the health-related goal for the user;
match the element of the expert rules data with an element of the fitness class data, wherein:
the fitness class data corresponds to outcomes associated with the fitness class; or
the fitness class data corresponds to preference factors associated with the fitness class;
determine recommendation data based on the element of the expert rules data matching with the element of the fitness class data, the recommendation data corresponding to a recommendation of the fitness class for the user; and
output, to the user device, the recommendation data.

2. The system of claim 1, the measurement by the wearable device comprising:
a biometric reading taken by the wearable device from the user, the measurement data comprising biometry data that corresponds to biometric information about the user; or
an activity reading taken by the wearable health monitoring device from the user, the measurement data comprising activity data that corresponds to activity engaged in by the user,
wherein the recommendation data is of the fitness class is based in part on the biometric reading or the activity reading.

3. The system of claim 1, the recommendation server configured to determine the health objective data:
directly based on the user data, wherein the user data explicitly includes the preference of the user or the health-related goal of the user;
inferentially based on the user data, the recommendation server further configured to:
generate a tag for the element of the user data, wherein the tag corresponds to the health information or the fitness information about the user;
determine the tag corresponds to generic data, the generic data comprising:
generic preference data that corresponds to a generic health- or fitness-related preference, wherein the generic preference data is stored on the recommendation server; or
generic goal data that corresponds to a generic health- or fitness-related goal, wherein the generic goal data is stored on the recommendation server; and
determine, based on the tag corresponding to the element of the user data and the generic data:
that the preference of the user is the generic health- or fitness-related preference; or
that the health-related goal of the user is the generic health- or fitness-related goal.

4. The system of claim 1, wherein:
the user health data comprises:
biometry data that corresponds to biometric information about the user;
vitals data that corresponds to demographic vitals of the user; or
health goal data that corresponds to a health goal for the user;
the user fitness data comprises:
user location data that corresponds to a location of the user;
fitness routine data that corresponds to a fitness routine of the user;
fitness goal data that corresponds to a fitness goal for the user;
preference data that corresponds to an exercise preference of the user; or
skill data that corresponds to a self-identified skill level of the user in a fitness class; and
the recommendation of the fitness class is based in part on the user health data or the user fitness data.

5. The system of claim 1, wherein:
the fitness class data comprises:
class location data that corresponds to a location of the fitness class;
schedule data that corresponds to a schedule for the fitness class;
instructor data that corresponds to information about an instructor of the fitness class;
class exercise data that corresponds to a type of exercise performed in the fitness class; or
difficulty data that corresponds to a difficulty level of the fitness class; and
the recommendation of the fitness class is based in part on the fitness class data.

6. The system of claim 1, wherein:
the user fitness data comprises:
user location data that corresponds to a location of the user;
location range data that corresponds to a preference of the user for a maximum distance from the location of the user to a location of the fitness class;
the fitness class data comprises class location data that corresponds to the location of the fitness class; and
the recommendation of the fitness class for the user is based in part on an actual distance between the location of the user and the location of the fitness class being less than the maximum distance.

7. The system of claim 1, wherein:
the fitness class server comprises a website server; or
the fitness class server comprises an application installed on the user device.

8. The system of claim 7, the recommendation server further comprising a data extraction application configured to:
retrieve the fitness class data from the website server; or
retrieve the fitness class data from the application on the user device.

9. A system, comprising:
an application server configured to:
receive user data, the user data comprising:
user health data that corresponds to health information about a user; or
user fitness data that corresponds to fitness information about the user;
receive fitness class data, the fitness class data corresponding to information about a fitness class;
communicate the user data or the fitness class data to a first receiving device;
a data server in communication with the application server, the data server configured to:
receive, from the application server:
the user data; or
the fitness class data;
store:
the user data;
the fitness class data; or
expert rules data that corresponds to a rule for making a health recommendation, the rule created by a health expert or a fitness expert;
communicate the user data, the fitness class data, or the expert rules data to:
the first receiving device; or
a second receiving device;
a recommendation server in communication with the application server or the data server, the recommendation server configured to:
receive, from the application server or the data server:
the user data;
the fitness class data; or
the expert rules data;
determine health objective data:
directly based on the user data; or
inferentially based on the user data;
match an element of the health objective data with a corresponding element of the expert rules data, wherein the element of the expert rules data corresponds to an activity for the user;
match the element of the expert rules data with an element of the fitness class data, wherein activity is performed in the fitness class; and
in response to the element of the expert rules data matching with the element of the fitness class data, determine recommendation data that corresponds to a recommendation of the fitness class for the user; and
communicate the recommendation data to:
the first receiving device;
the second receiving device; or
a third receiving device.

10. The system of claim 9, further comprising:
the user device wherein:
the user device configured to receive input from a user; and
the user device is the first receiving device, the second receiving device, or the third receiving device; and
a wearable device in communication with the user device or the application server, the wearable device configured to take a measurement from the user, wherein the measurement comprises:
a biometric measurement; or
an activity measurement.

11. The system of claim 10, wherein:
the user health data comprises:
input data corresponding to the input from the user at the user device; or
biometry data corresponding to the biometric measurement taken by the wearable device; or
the user fitness data comprises:
the input data; or
activity data corresponding to the activity measurement taken by the wearable device.

12. The system of claim 10, the application server further configured to:
receive the recommendation data from the recommendation server, the recommendation server further configured to output the recommendation data to the application server;
convert the recommendation data from a raw format to a user-readable format; and
communicate the recommendation data in the user-readable format to the user device or the wearable device, wherein the user uses the recommendation data to select the fitness class.

13. The system of claim 9, the application server further configured to retrieve the fitness class data from a third-party data resource that contains information about the fitness class, wherein the third-party data resource comprises:
a public website;
a private website; or
a fitness application installed on a user device.

14. The system of claim 9:
the data server comprising a relational database that relates activity data with outcome data, wherein:
the expert rules data comprises the activity data and the outcome data;
the activity data corresponds to a type of activity; and
the outcome data corresponds to physiological effects of participating in the activity; and
the recommendation server further configured to:
match the element of the health objective data with the element of the rule data by inferring the health objective data corresponds to the outcome data; or
match the element of the expert rules data with the element of the fitness class data by inferring the fitness class data corresponds to the activity data.

15. The system of claim 14, wherein the type of activity comprises:
a physical exercise activity;
a nutrition activity; or
a mindfulness activity.

16. A system, comprising:
a memory device configured to store:
user data comprising:
user health data that corresponds to health information about a user; or
user fitness data that corresponds to fitness information about the user;
fitness class data that corresponds to information about a fitness class; and
expert rules data that corresponds to an action for accomplishing a health objective;
a processing device in communication with the memory device, the processing device configured to:
determine health objective data:
directly based on the user data; or
inferentially based on the user data;
match an element of the health objective data with a corresponding element of the expert rules data, wherein the element of the health objective data and the element of the expert rules data are matched directly or inferentially;
match the element of the expert rules data with an element of the fitness class data, wherein:
the fitness class data corresponds to an aspect of the fitness class that aligns with the health objective of the user based on the expert rules data; and
the element of the expert rules data and the element of the fitness class data are matched directly or inferentially;
determine recommendation data that corresponds to a recommendation of the fitness class for the user; and
output, to a receiving device, the recommendation data.

17. The system of claim 16, further comprising:
a user device in communication with the memory device or the processing device, the user device configured to:
receive a user input;
generate input data corresponding to the user input, the input data being:
a first element of the user health data; or
a first element of the user fitness data;
communicate the input data to the memory device or the processing device;
receive the recommendation data, wherein the user device is the receiving device; and
display the recommendation based on the recommendation data; or
a measurement device in communication with the memory device or the processing device, the measurement device configured to:
take a physiological measurement from the user; and
generate measurement data based on the physiological measurement, the measurement data being:
a second element of the user health data; or
a second element of the user fitness data; and
communicate the measurement data to the user device, the memory device, or the processing device.

18. The system of claim 16, further comprising a location device in communication with the memory device or the processing device, wherein:
the location device is configured to output user location data that corresponds to a location of the user;
the fitness class data comprises class location data that corresponds to a location of the fitness class;
the processing device is further configured to correlate the user location data with the fitness class data; and
the recommendation data is further based on the user location data correlating with the fitness class data.

19. The system of claim 18, wherein the user location data correlates with the fitness class data when the location of the fitness class is less than or equal to a maximum distance from the location of the user.

20. The system of claim 16, wherein the fitness class is recommended to the user based on a goal of the user represented in the user data such that participation by the user in the fitness class helps the user accomplish the goal of the user.
